(19) United States
(12) Patent Application Publication
(10) Pub. No.: US 2025/0009851 A1
(43) Pub. Date: Jan. 9, 2025

(54) SECRETED SPLICING VARIANT OF MAMMAL KLOTHO AS A MEDICAMENT FOR COGNITION AND BEHAVIOUR IMPAIRMENTS

(71) Applicants: Universitat Autonoma de Barcelona, Bellatera (Cerdanyola del Valles) (ES); Fundació Institució Catalanà de Recerca i Estudis Avançats, Barcelona (ES)

(72) Inventors: Miguel Chillon Rodriguez, Barcelona (ES); Anna Masso Chacon, Vilanova i la Geltrú (ES); Assumpció Bosch Merino, Sant Cugat del Vallès (ES)

(73) Assignees: Universitat Autonoma de Barcelona, Bellatera (Cerdanyola del Valles (ES); Fundació Institució Catalanà de Recerca i Estudis Avançats, Barcelona (ES)

(21) Appl. No.: 18/773,164

(22) Filed: Jul. 15, 2024

Related U.S. Application Data
(63) Continuation of application No. 15/777,456, filed on May 18, 2018, now Pat. No. 12,036,268.

(30) Foreign Application Priority Data
Nov. 19, 2015 (EP) 15195470.8

(51) Int. Cl. A61K 38/47; A61K 9/00; A61K 45/06; A61K 48/00; A61P 25/28; C07K 16/40; C12N 9/24

(52) U.S. Cl. CPC A61K 38/47; A61K 9/0085; A61K 45/06; A61P 25/28; C07K 16/40; C12N 9/2402; C12Y 302/01031; A61K 48/00; C12N 2710/10043

(57) ABSTRACT

The invention discloses using secreted splicing variant of mammal Klotho (s-KL) as an agent for the prevention and/or treatment of cognitive and/or behaviour impairments. It also refers to gene constructs and expression vectors useful in gene therapy for the delivery of said s-KL variant to the central nervous system of a mammal, in particular a rodent or a human. Pharmaceutical compositions comprising either the protein s-KL or any gene construct for expressing the protein in the CNS are also disclosed.

The present invention relates to the field of medical approaches for preventing and/or treating cognition impairment and behaviour impairments in patients suffering from diseases related to memory loss, and to learning difficulties, and/or with neurodegenerative and/or neuropathological diseases.

### BACKGROUND ART

It is widely known that memory is frequently lost along years and that some aspects can be changed leading to a loss of memory efficiency. Examples of these aspects include the difficulty of maintaining concentration to more than one thing at the same time; difficulties in learning new things that require an effort, and slow old information recuperation. Spoken abilities, information processing, problem resolution, working memory, long-term memory and spatial memory and ability are reduced with age, meanwhile expertise and cognitive abilities, such as semantic memory (vocabulary), world knowledge and implicit memory remain stable with age or even are bettered if no pathologies or neurodegeneration is present.

In connection with the neurodegenerative and/or neuropathological diseases, many of them related with aging, imply also cognitive impairments. Examples of these include Alzheimer's disease, Parkinson's disease, Huntington's disease, depression and schizophrenia.

In addition, with aging or in case of neurodegenerative and/or neuropathological diseases also behaviour impairments are observed, such as an increase in disorientation, anxiety episodes to unknown situations or places or even to the disorientation itself, as well as irrational fear.

All these cognitive and behaviour impairments are the major aim not only in the treatment of the symptomatology of many of the neurodegenerative and/or neuropathological diseases, but also in the prevention of cognition and behaviour problems during aging.

Among the several and complex mechanisms associated with memory loss during aging or in case of neuropathology or neurodegeneration, the role of the mammal protein Klotho has been studied.

In three independent human assays it was discovered that some mutations in klotho allele, namely the KL-VS klotho allele in vitro promoting an increase of klotho secretion, could get better results in cognitive tests in an independent manner of the age of the subjects. These data derive from the document of Deary et al., “Klotho genotype and cognitive ability in childhood and old age in the same individuals”, Neurosci Lett-2005, vol. 378 (1), pp. 22-27.

Klotho is a protein detected primarily in the distal convoluted tubule of the kidney, parathyroid hormone-secreting cells and choroid plexus epithelium of the brain. To a lesser extent a-klotho gene is also expressed in heart, skeletal muscle, urinary bladder, placenta, pancreas, testes, ovaries, colon and inner car. Several studies in mice have revealed that the mutation of the single gene a-klotho on chromosome 13, induces a process of accelerated aging (Sec. Kuro-o, et al., Mutation of the mouse klotho gene leads to a syndrome resembling ageing. Nature-1997, vol. no. 390, pp.: 45-51; and Wang, Y.et al, “Current understanding of klotho”, Ageing Res Rev-2009, vol. no. 8, pp.: 43-511) with mimicking typical symptoms such as atherosclerosis, osteoporosis/osteopenia, ectopic calcification in various tissues, emphysema, cognitive problems and infertility. Moreover, knockout a-klotho (klVkl″) mice showed dramatically reduced survival with prematurely around 2 months of age (Kuro-o, et al, supra). In contrast, transgenic mice overexpressing this gene had between 30 to 40% greater life expectancy. Similarly, several studies indicate that human klotho gene polymorphisms also affect the longevity and appearance of disorders associated with aging. In mice and humans, a-klotho gene encodes a transcript of a 5.2 kb. In the third exon there is an alternative splicing donor site that can generate two different transcripts: one encoding a transmembrane form (full transcript or full-length, 1014 amino acids) and the other a secreted form of the protein (half transmembrane transcript, 550 amino acids). The full-length transcript encodes a single pass transmembrane protein with a molecular weight of approximately 130 kDa (m-KL). The protein contains three domains: a short transmembrane domain at the C-terminal, an extracellular domain composed of two internal repeated sequences of about 550 amino acids called KL1 and KL2 respectively, and a very short intracellular domain of 10 amino acids. The transcript from alternative splicing generates a truncated form of the protein (s-KL) that is formed solely by the KL1 domain, with an approximate weight of 70 kDa. This alternative mRNA includes a specific secretion signal consisting of 15 amino acid tail that is not found in the m-KL transcript, and for this reason is also called the secreted isoform of klotho, s-KL, or the secreted splicing variant of klotho protein (See Matsumura et al., “Identification of the human klotho gene and its two transcripts encoding membrane and secreted Klotho protein”, Biochem Biophys Res Commun-1998, vol. No. 242, pp.: 626-630). However, there is some controversy regarding this protein (s-KL) because it has not been detected in body fluids using antibodies. It has been specifically detected at mRNA levels, but not at protein level since the available antibodies cannot differentiate it from the KL1 domain cleaved from the transmembrane Klotho.

In addition, the extracellular domain of the transmembrane form can be cleaved by metalloproteinases ADAM10 and ADAM17 resulting in another form of soluble Klotho of about 130 kDa (abbreviated p-KL for proteolyzed membrane isoform), which has been detected in serum, urine, and cerebrospinal fluid. Moreover, two recent studies indicate that there is a second recognition site for the proteases ADAM 10 and 17 located between the KL1 and KL2 domains, which generates two new 70 kDa isoforms, one contained the KL1 domain only (like the one generated from alternative splicing but without the specific amino acid tail), and the other one contained the KL2 domain. Thus, Klotho protein might enter the circulatory system through two main mechanisms: (a) from an alternative splicing (s-KL), and (b) by proteolytic cleavage mediated by ADAM 10 and 17. However, it is unknown the percentage that each of these events occurs.

With the aim of elucidating the role of Klotho as anti-aging factor and as agent improving learning and memory skills, systemically over-expressing Klotho transgenic mice have been obtained. Dubai et al. 2014 discloses that the variant of the human klotho gene KL-VS is associated with enhanced cognition in heterozygous carriers. This allele increased klotho levels in serum. Then they analyzed transgenic mice with global overexpression of full-length transmembrane klotho, performing better than controls in multiple tests of learning and memory (Dubai et al., “Life Extension Klotho Enhances Cognition”, Cell Reports-2014, vol. 7, pp.: 1065-1076). Another transgenic mouse also analyzed by Dubai et al., is particularly focused on Alzheimer's disease and it shows that elevating klotho expression decreases premature mortality, network dysfunction, cognitive deficits and behavioural abnormalities in human amyloid precursor protein (hAPP) transgenic mice, and all without altering the levels of hAPP (Dubai et al., Life Extension factor Klotho Prevents Mortality and Enhances Cognition in hAPP Transgenic Mice”, The Journal of Neuroscience—2015, vol. 35/6, pp.: 2358-2371). In said transgenic models it is likely evaluated the effect of the full-length transmembrane protein with a molecular weight of approximately 130 kDa (m-KL), and if any of the other isoforms are contributing to the effect, specially the processed Klotho (p-KL) isoform, this cannot be determined from the experimental procedure used by authors since current available detection systems are not capable of distinguishing among the different klotho isoforms. Although transgenic animals over-expressing a protein of interest may be good models for the analysis of its effects, they also imply the inconvenient of having this protein expressed ubiquitously (body and brain), and data may in addition be made up by the genetic background of the transgenic animal.

Another document showing the potential role of klotho in a mouse model of senescence (named SAMP8) is the one of Kuang et al., “Klotho upregulation contributes to the neuroprotection of ligustilide (LIG) in an Alzheimer's disease mouse model”, Neurobiology of Aging—2013, pp. 1-10. In this document ligustilide is proposed as treatment in Alzheimer's disease (AD) for reducing memory deficits. The data show that chronic administration of LIG prevents the development of AD-like neuropathologies and memory impairment in aging. In addition data suggest that the likely underlying mechanism involve Klotho up-regulation. Thus klotho protein is suggested as therapeutic target for age-related AD. According to the experimental methodologies disclosed by Kuang et al. (use of antibody of Santa Cruz Biotechnology, Santa Cruz, CA, USA to detect Klotho protein), no distinction among the acting protein isoform is possible (m-KL, p-KL). When Kuang et al. refers to “secreted klotho”, it does not refer to the secreted splicing variant of mammal Klotho but to other soluble forms of klotho. In fact, at that time, and until the findings of the inventors of the present invention, the alternatively secreted Klotho isoform was considered a very minor variant, not expressed (or expressed at negligible levels). In fact as reported by Forster et al.: “A klotho form containing both the KL1 and KL2 domains has been detected in both serum and cerebral spinal fluid (CSF), and has been interpreted as a proteolytic fragment of m-KL. The existence of a circulating klotho species that exactly corresponds to the peptide which would be produced from the alternatively spliced mRNA has not been reported in serum or CSF” (Forster et al. “Vitamin D Receptor Controls Expression of the Anti-aging Klotho Gene in Mouse and Human Renal Cells” Biochem Biophys Res Commun. 2011 October 28; 414 (3): 557-562). Albeit the several approaches for facing cognitive impairments due to aging or some neurodegenerative diseases, there is still need of alternative treatments for ameliorating cognitive and behaviour maintenance.

### SUMMARY OF THE INVENTION

The present invention results from inventor's determination of the real expression at protein level (not only as mRNA) of the splicing variant of mammal klotho protein in mouse wild-type brain tissue. Using a self-made antibody raised against a peptide amino acid sequence comprised in the last twenty amino acids of the C-terminal end of the mice s-KL, inventors detected that in the whole brain of wild-type mice (C57BI6 of Harlan Laboratories BV), and in some specific parts of the brain the expressed isoform of Klotho protein was an isoform having a molecular weight near 70 KDa, likely s-KL but with posttranslational modifications. In addition, inventors found (data not shown) that expression levels of this protein were lowered during aging and with AD progression.

Further assays revealed that in brain this splicing variant could be determined at protein level. Moreover, the inventors have found that the secreted klotho isoform is at least ten times more abundant in the brain than in the kidney suggesting that the two isoforms may have different functions, and that s-KL activity is the isoform with an important role in the nervous system.

The inventors have herein studied the functional relevance at behavioural level, of modifying s-KL levels in the aging brain. They used AAVrhI O vectors to deliver and sustained expression of s-KL in adult and middle-aged wild-type C57BL/6J males. This study demonstrates for the first time in vivo, that six months after a single injection of s-KL into the CNS, long-lasting and quantifiable enhancement of learning and memory capabilities are found. More importantly, cognitive improvement is observable in 18-months-old mice treated once, at middle-age. These findings demonstrate the therapeutic potential of s-KL as a treatment for cognitive decline.

In summary, it is herein demonstrated that local overexpression of the secreted Klotho isoform locally in the brain after a single administration of an AAV-sKL vector protects against age-dependent memory deficits, being these effects long-lasting and quantifiable in old animals, and therefore suggesting a therapeutic potential of s-KL for dementia. Local expression of s-KL in the CNS improves cognitive performance in aged mice. s-KL expression in the CNS does not affect body weight or sensorimotor skills. Cognitive improvement is observed in 18-months-old mice treated once, at middle-age. As important, specific s-KL inhibition in hippocampus by specific shRNA-sKL impairs cognitive performance. Therefore, the inventors are able to demonstrate the key implication of secreted Klotho in memory formation, by using a dual strategy based on: (1) sKL overexpression to increase memory performance, and (2) sKL inhibition to reduce memory performance.

Thus, it is believed that the inventors have found for first time, that the klotho transcript produced by alternative splicing generates a protein of 70 KDa which they demonstrate that it is stable, and that this isoform is more abundant in brain that in other parts of the body. Moreover, it is believed that is the first time that secreted klotho is directly administered in vivo, not expressed by means of a transgenic model. As said before, the administration of s-KL leads to an amelioration of cognitive and behaviour faculties. Thus, the inventors provide the use of s-KL as a new treatment for cognitive and/or behaviour impairments, and/or with neurodegenerative and/or neuropathological diseases. This represents a more specific and CNS-oriented treatment for these diseases, since it is demonstrated herein that s-KL is the most prevalent isoform in brain parts related to cognition and behaviour natively expresses s-KL.

Prior art, as explained before, relates to transgenic mice systemically overexpressing the full-length coding region of klotho. However, full-length coding region may allow not only transcription of the transmembrane isoform, but also the alternatively splice isoform. Therefore it is not possible to dilucidate whether the amelioration of learning and memory effects reported are due to one or the other klotho isoforms. On the other hand, despite the inventors observed that both klotho isoforms seem to participate in memory performance, interestingly, overexpression of m-KL may affect in a non-specifically way other pathways unknown at this moment, resulting in relevant side effects, such as the ones described in Example 2C of this description, while specific overexpression of s-KL does not. In addition, differently from the transgenic mice of the prior art, the present approach does not alter natural Klotho levels from the birth of the animal, which may induce the apparition of compensatory effects, until key stages in the aging process are reached. Then levels are specifically modified in the brain through administration of AAVrhI O vectors with neuronal tropism.

Thus, the inventors propose as a first aspect the secreted splicing variant of mammal Klotho protein or the nucleic acid sequence coding therefor for use in the prevention and/or treatment of cognitive and/or behaviour impairment, and/or with neurodegenerative and/or neuropathological diseases in a mammal.

This aspect can also be formulated as the use of s-KL as defined above for the manufacture of a medicament for the prevention and/or treatment of cognitive and/or behaviour impairment, and/or with neurodegenerative and/or neuropathological diseases. The present invention also relates to a method for the treatment or prevention of cognitive and/or behaviour impairment, and/or with neurodegenerative and/or neuropathological diseases, comprising administering a therapeutically effective amount of s-KL protein or of nucleic acid sequence coding therefor as defined above, together with pharmaceutically acceptable excipients or carriers, in a subject in need thereof, including a human.

The splicing variant s-KL of Klotho protein appears, as above exposed, disclosed in Matsumura. In addition, other documents refer also to this splicing variant. As a way of example, the document Shiraki-lida et al., “Structure of the mouse klotho gene and its two transcripts encoding membrane and secreted protein”, FEBS Letters-1998, vol 424, pp.: 6-10, discloses a supposed s-KL1 secreted protein detected at mRNA level. On the other side, the document of Imura et al., “Secreted Klotho protein in sera and CSF: implication for post-translational cleavage in release of Klotho protein from cell membrane”, FEBS Letters-2001, vol 565, pp.: 143-147 refers also to a putative secreted isoform that is not detected in extracellular fluid. None of these documents points to any role of the splicing variant s-KL of klotho protein.

The term “secreted splicing variant of mammal Klotho” abbreviated as “s-KL”, refers to the protein resulting from the transcript from alternative splicing, which generates a truncated form of the protein (s-KL) that is formed solely by the KL1 domain, with an approximate weight of 70 kDa. This alternative mRNA includes a specific secretion signal consisting of 15 amino acid tail that is not found in the m-KL transcript, and for this reason is also called the secreted isoform of klotho, s-KL, or the secreted splicing variant of klotho protein. s-KL is different from other forms of soluble klotho, namely p-KL, p-KL1 and p-KL2. In this description, m-KL stands for the full-length transmembrane form; p-KL stands for the soluble proteolyzed klotho, which is generated by cleavage of the m-KL; and p-KL1 and p-KL2 stand for the soluble klotho forms consisting of the KL1 domain and the KL2 domain of p-KL. m-KL comes from the full-length transcript encoding a single pass transmembrane protein with a molecular weight of approximately 130 kDa (m-KL). The protein contains three domains: a short transmembrane domain at the C-terminal, an extracellular domain composed of two internal repeated sequences of about 550 amino acids called KL1 and KL2 respectively, and a very short intracellular domain of 10 amino acids. The extracellular domain of the transmembrane form can be cleaved by metalloproteinases ADAM10 and ADAM17 resulting in another form of soluble Klotho of about 130 kDa (abbreviated p-KL for proteolyzed membrane isoform. Moreover, there is a second recognition site for the proteases ADAM10 and 17 located between the KL1 and KL2 domains, which generates two new 70 kDa isoforms, one contained the KL1 domain only (like the one generated from alternative splicing but without the specific amino acid tail), and the other one contained the KL2 domain. However, it has not been demonstrated in vivo that p-KL is proteolyzed into p-KL1 and p-KL2.

In a second aspect the invention relates to a gene construct comprising a nucleic acid sequence coding for the secreted splicing variant of mammal klotho protein (s-KL) operatively linked to an expression promoter.

Another aspect of the invention is an expression vector with central nervous system tropism comprising the gene construct as defined above, thus comprising a nucleic acid sequence coding for the secreted splicing variant of mammal klotho protein (s-KL) operatively linked to an expression promoter.

Yet another aspect of the invention is a pharmaceutical composition comprising a therapeutically effective amount of the secreted splicing variant of mammal klotho protein and/or the gene construct as defined above, and/or the vector as defined above, together with one or more pharmaceutically acceptable excipients or carriers.

It is also an aspect of the invention the secreted splicing variant of mammal klotho protein or the nucleic acid sequence coding therefor, for use in combination therapy for the prevention and/or treatment of cognitive and/or behaviour impairment, and/or with neurodegenerative and/or neuropathological diseases in a patient, wherein the protein or the nucleic acid sequence coding therefor is to be administered in combination with another active agent for the same indication.

It is also an aspect of the invention a combination of the secreted splicing variant of mammal klotho protein and/or the nucleic acid construct as defined above, and/or the vector as defined above with another active agent for use in the prevention and/or treatment of cognitive and/or behaviour impairment, and/or with neurodegenerative and/or neuropathological diseases. The combination relates to a pharmaceutically composition or preparation for administration of s-KL with any other active agent, which can be done separately, in any order, within a therapeutically effective interval, or for the simultaneously administration of the active agents.

Hereinafter are described the advantages derived from the invention and the results obtained from the experimental procedures provided below.

The Open Field test was used to study whether s-KL is able to affect locomotion, exploratory activity, emotionality, neophobia and anxiety like behaviors. Briefly, the inventors found that regardless of age, the animals overexpressing s-KL show mild hyperactivity, an increased locomotor activity when compared to control animals, and therefore, that the administration of s-KL in the CNS seems to reverse, at least partially, the gradual decline in locomotor activity observed around 12 months of age. In addition, the latencies of movement (an indicator of behavioral inhibition as a result of neophobia or anxiety) tended to be shorter in s-KL treated animals, indicating a higher disinhibition, although this effect reached statistical significance only in middle-age animals (6->12 setting), but not in old animals (12->18 setting). Consistently, the inhibition of s-KL expression mediated by shRNA-sKL had an opposite effect. Thus, some animals of the shRNA-sKL group exhibited behavioral inhibition at the start of the test, showing freezing or petrification, and latencies in leaving the central area far superior to mice overexpressing s-KL.

Next, to evaluate the effect of s-KL overexpression on the working memory the T-maze test was used. Again, the results show that administration of s-KL, regardless of age, improves cognitive abilities since s-KL treated mice make less errors in solving the task as compared to control animals. These results concur with the increase in s-KL levels found in the prefrontal cortex of the (12->18 months setting) animals. Given that in mice, deficits in working memory appear around 24 months of age, Klotho seems to be acting as an enhancer of cognitive functions.

Finally, the Morris Water Maze test was used to assess the effect of s-KL overexpression/inhibition on visual perceptual learning and memory and learning abilities. The results from both experiments showed again that s-KL treated mice are more efficient in solving the task and they learn faster than the control animals, indicating that s-KL significantly improves long-term memory in mice. In contrast, animals injected with shRNA-sKL showed problems in learning the task and swam a greater distance to reach the platform. Moreover, these opposite effects were also observed in the final memory tests (24 hours after the last training). Animals overexpressing s-KL prioritized the search in the quadrant where the platform was previously located, whilst shRNA-sKL treated animals showed no particular preference for the training quadrant, suggesting memory and/or learning problems. As expected, when compared to controls, quantification of s-KL levels 6 months after treatment (both, mRNA and protein) showed a very strong positive correlation with cognitive capacities, being statistically higher in AAV/s-KL treated mice and lower in AAV/shRNA-sKL treated mice. This pattern was observed regardless of whether animals were inyected in adulthood (6->12 months setting) or middle age (12->18 months setting).

In summary, this study provides new evidence indicating an important role for s-KL in cognitive functions, with reduced levels in hippocampus being associated to low cognitive performance. The study also demonstrates that a single icv injection of s-KL into the CNS has great potential as a long-lasting and quantificable agent to stimulate cognitive skills, even, protecting age-dependent cognitive decline when mice were treated at old ages. To the inventors' knowledge, these are the first data obtained in vivo, in which the action of only the secreted Klotho protein improves the learning and memory capabilities of old animals when treated in adulthood. Furthermore, taking into account that these experiments were performed in naive aged animals, the results suggest s-KL may have therapeutic potential for dementia. This represents a promising new therapeutic approach for neurodegenerative disorders such as Alzheimer's Disease or Multiple Sclerosis among others.

### BRIEF DESCRIPTION OF THE DRAWINGS

FIG. 1 is a schematic view of the procedure for constructing adeno-associated virus as vectors carrying a plasmid with the gene construct of the invention for expressing the different variants of klotho protein. In (A) the pUC57-KL synthetic plasmid (GenScript, USA) is partially represented showing an insert coding for klotho protein and indicating the restriction sites. KL1 indicates the sequence coding for KL1 domain and comprises a contiguous square representing the sequence coding for the tail of amino acids only present in the secreted splicing variant of klotho s-KL. KL2 represents the sequence coding for KL2 domain of klotho. In panel (B) it is schematically viewed the strategy to obtain from the synthetic pUC57-pKL, the pGG2-sKL plasmids carrying the sequence coding for the klotho protein. This plasmid is then introduced in 293-AAV cells (Stratagene). AAV-s-KL (or AAVrh 10-s-KL) designates the adeno-associated virus carrying the plasmid with the gene construct coding for s-KL. This figure is related to Example 1.

FIG. 2 (A, B) shows, respectively, the total distance (cm) in open field test made by Null, m-KL or s-KL 18 months old mice (panel A); the total distance (cm) made per minute for each type of mouse (panel B), recorded during one minute (in X-axis, MIN is the minute 1 to 5 of assay, circles for Null, squares for m-KL and triangles for s-KL). This figure is related to Example 2C.

FIG. 3 shows the results of a T-maze test performed in control mice (squares), and s-KL mice (circles). Percentage of success in the free choice test distributed along the different days of the assay (from day 1 to day 3). This figure is related to Example 2D.

FIG. 4 depicts the results of a Morris Water Maze test performed in s-KL mice (circles), and Null mice (squares). FIG. 4 (A) depicts per each place task (PT) test performed 1 to 4, the mean speed (in cm/s) to get the platform. FIG. 4 (B) indicates the mean distance (in cm) to get the platform. The test was performed with 18 months old mice. This figure is related to Example 2D.

FIG. 5 is a graphic showing the results of a long-term memory test performed 24 hours after a cue test part of the Morris Water Maze test. The test was performed with 18 months old mice. In Y-axis it is recorded the percentage of distance (%) in relation to the total done in several squares of the swimming pool. Left bar in each group (Null, or s-KL) is the percentage of distance in the square where previously the platform was disposed (PTf square); second bar in each group shows the percentage of distance in the opposite square (Opos PTf), third bar is the percentage of distance made in the square at right of the platform (R PTf), and right bar in each group is the percentage of distance made in the square at the left of the platform (L PTf). This figure is related to Example 2D.

FIG. 6 shows relative expression in relation to controls of s-KL in several brain parts or sections (Prefrontal cortex, PFC; cortex, C; hippocampus, H; and cerebellum, CB) of AAV-treated animals. This figure is related to Example 2E.

FIG. 7 (A, B) shows, respectively, the total distance (cm) in open field test made by s-KL, shRNA-sKL and Control, 12-months old mice (panel A); the total distance (cm) made per minute for each type of mouse (panel B), recorded during one minute (in X-axe, MIN is the minute 1 to 5 of assay, squares for Null, triangles for shRNA-sKL and circles for s-KL); and different parameters to evaluate the exploratory activity in the open field test, (panel C). This figure is related to Example 3B.

FIG. 8 depicts the results of a Morris Water Maze test performed in in 12-months old mice. s-KL mice (circles), shRNA-sKL mice (triangles), and Null mice (squares). FIG. 8 (A) depicts per each place task (PT) test performed 1 to 4, the mean latency (in seconds, s) to get the platform. FIG. 8 (B) indicates the mean distance (in cm) to get the platform. The test was performed with 18 months old mice. FIG. 8 (C) shows the results of the Morris Water Maze memory test performed in 12-months old mice, and performed 24 hours after the cue test part of the test. In Y-axis it is recorded the percentage of distance (%) in relation to the total distance done in several squares of the swimming pool. Left bar in each group (s-KL, shRNA-sKL and Control) is the percentage of distance in the square where previously the platform was disposed (PTf square); second bar in each group shows the percentage of distance in the opposite square (Opos PTf), third bar is the percentage of distance made in the square at right of the platform (R PTf), and right bar in each group is the percentage of distance made in the square at the left of the platform (L PTf). This figure is related to Example 3C.

FIG. 9 is a Western blot image of an assay conducted to detect klotho protein in wild-type mice (C57BI6) tissues with a commercial antibody (KM2076, of Cosmobio Japan): Kidney, Whole brain and particular brain sections prefrontal cortex (CPf), in cortex (Cx), cerebellum (CB), and hippocampus (HC). Klotho protein in brain had a molecular weight between 70 and 100 KDa, meanwhile in kidney the protein has a molecular weight near 130 KDa. This figure is related to Example 4.

FIG. 10 (A-D) shows the analysis of the s-KL protein by Western-blot using a rabbit anti-mouse antibody (self-made and named Ab K113), raised specifically against s-KL. Prefrontal cortex (PfCx, panel (A)), cortex (CX,panel (B)), hippocampus (HC, panel (C)), and cerebellum (CB, panel (D)) of 6 and 18 months old C57BI/6 mice (YOUNG 6M, AGED 8M). Actine (42 kDa) was used to normalize the amount of protein analyzed. Samples were quantified by densitometry using ImageJ software, the public domain, Java-based image processing program developed at the National Institute of Health. For each brain area analysed, Fold-change from 18 months-old mice relative to those obtained from 6 months-old mice are depicted in bar diagrams.

FIG. 11. s-KL and m-KL expression levels in AAV-treated mice. s-KL levels were quantified in the hippocampus of s-KL, shRNA-sKL and null treated animals 6 months after hippocampal administration. mRNA levels of s-KL and m-KL transcripts were normalized respect the values obtained in control animals. This figure is related to Example 5.

### DETAILED DESCRIPTION OF THE INVENTION

All terms as used herein, unless otherwise stated, shall be understood in their ordinary meaning as known in the art. Other more specific definitions for certain terms as used in the present application are as set forth below and are intended to apply uniformly throughout the description and claims unless an otherwise expressly set out definition provides a broader definition.

A “gene construct” according to the invention can also be named as an “expression cassette”. It refers to a polynucleotide sequence including in turn a sequence coding for a protein of interest, which is operatively linked to a expression promoter, said promoter controlling expression of the sequence coding for the protein. For “operatively linked” is to be understood that the sequence coding for the protein is disposed after the sequence of the promoter (in the 5′-3′ direction), or near the promoter in case restriction sites are included, or other stabilizing elements of the gene construction are present. The gene constructs (expression cassette) may also comprise small fragments with useful sequences to adapt it to more complex expression systems (vectors, plasmids), or a polyadenylation tail disposed after the sequence coding for the protein of interest. The expression cassette itself is also a expression system, being vectors or plasmids further used to protect the gene construct, or to promote entrance to cells in case of viral vectors. The “promoters” are of DNA regions that initiate transcription of a particular genes. Promoters are located near the transcription start sites of genes, on the same strand and upstream on the DNA (towards the 5′ region of the sense strand). Promoters can be about 100-1000 base pairs long. A “constitutive promoter” is a promoter that is active in all circumstances in the cell, contrary to others that are regulated, becoming active in the cell only in response to specific stimuli, such as “inducible promoters”. Other promoters are tissue or cell-specific, such as “neuron-specific promoters”. For “polynucleotide sequence” it is to be understood as a nucleic acid molecule (DNA or RNA) comprising deoxyribonucleotides or ribonucleotides. Nucleic acid can be single or double stranded, and it includes, but it is not limited to, nucleotide sequences coding for polypeptides.

The term “adeno-associated virus (AAV)” as used herein refers to a viral vector that infects both dividing and quiescent primate (and human) cells. Because they seem to lack any pathogenic effects, and usually integrate in the same place of the genome (the AAVSI site, in chromosome 19), this viral vectors can safely be used to transduce foreign DNA into human cells in gene therapy applications. An “expression vector”, otherwise known as an “expression construct”, is usually a plasmid or virus designed for protein expression in cells. The vector is used to introduce a specific gene into a target cell, and can commandeer the cell's mechanism for protein synthesis to produce the protein encoded by the gene. Expression vectors are the basic tools in biotechnology for the production of proteins. An expression vector has features that any vector may have, such as an origin of replication, a selectable marker, and a suitable site for the insertion of a gene such as the multiple cloning site.

The “percentage of homology” between two amino acid sequences is to be understood as the percentage of the sequence positions identical or replaced with other amino acids with lateral chains of similar features (i.e. polar, non-polar, with amino groups, with —SH groups, that is, amino acids the same class), according to the broadly accepted classifications known by an expert in the field. The “percentage of identity” between two amino acid sequences is to be understood as the percentage of the sequence positions with identical amino acids. The percentage of homology and of identity between sequences may be calculated by means of “sequence alignment”. The sequence alignment may be local or global. In the sense of the present invention the percentage of homology and of identity will be calculated, preferably, over a global alignment, among the entire sequence or an entire active fragment of the sequence. Global alignments are more useful when the sequences are similar and have approximately the same size (long). There are several algorithms available in the state of the art for performing these global alignments. There are also bioinformatics tools using such algorithms to obtain the percentage of identity and homology between sequences. As an example, global alignment between sequences may be performed by means of the well-known GGSEARCH or GLSEARCH software. The identity between two amino acid sequences is preferably determined by using the BLASTP algorithm disclosed in Altschul, S. F., et al. “Gapped BLAST and PSI-BLAST: a new generation of protein database search programs”, Nucleic Acids Research-1997, Vol. No. 25, pp.: 3389-3402, and NCBI http://www.ncbi.nlm.nih.gov/BLAST.

As will be depicted in the examples below, brain injection to mice of expression vectors comprising gene constructs with a nucleic acid sequence coding for the secreted splicing variant of mammal klotho protein (s-KL) operatively linked to an expression promoter, lead to an amelioration of cognitive and behaviour functions when these mice were submitted to several tests. These data prove that s-KL can be used in the treatment and/or prevention of cognitive and/or behaviour impairment, and/or with neurodegenerative and/or neuropathological diseases.

The invention encompasses the administration of both the s-KL as protein or as nucleic acid sequence coding therefor, this last in a suitable form to be administered, such as in the form of a gene construct and/or expression vector. Thus, “s-KL” herein means both forms of s-KL.

In a particular embodiment, the cognitive and/or behaviour impairment is associated with aging. The term “associated with” means that cognitive and/or behaviour impairment takes place when mammals, in particular human, get older (aging), such as in senile dementia, and also may take place in some neurodegenerative and/or neuropathological diseases, being cause or consequence of the physiological parameters also affected in said diseases. Senile dementia is related to a condition appearing due to the natural non-pathological aging, and it implies many cognitive and/or behaviour impairments, such as balance problems, tremors, memory distortions, anxiety, depression, apathy, agitation and irritability. Senile dementia may also be associated with neurodegenerative diseases as Alzheimer's disease.

In a particular embodiment the cognitive and/or behaviour impairment associated with aging is one manifested in senile dementia.

Thus, in a particular embodiment of the first aspect of the invention, the s-KL is for use in the prevention and/or treatment of a cognitive impairment selected from the group consisting of learning and memory problems. More particularly, is for use in learning impairments, in particular learning procedure impairments, and memory problems, in particular memory losses, impairment of working memory, and impairment of long-term memory, the latter including spatial and episode memory impairments.

In another particular embodiment, optionally in combination with any embodiment above or below, s-KL is for use in the prevention and/or treatment of a behaviour impairment selected from anxiety and agoraphobia.

Also in another particular embodiment, optionally in combination with any embodiment above or below, s-KL is for use in the prevention and/or treatment of cognitive and/or behaviour impairment associated to neurodegenerative and/or neuropathological diseases selected from the group consisting of Alzheimer's disease, Parkinson's disease, Huntington's disease and Amyotrophic lateral sclerosis, Dementia with Lewy bodies, Creutzfeldt-Jakob disease, Multiple Sclerosis, and Ataxia telangiectasia, post stroke dementia, post-traumatic dementia, senile dementia, and craniocerebral trauma.

In a more particular embodiment s-KL is for use in the prevention and/or treatment of cognitive and/or behaviour impairment associated to Alzheimer's disease. In particular it is for use in the prevention and/or treatment of anxiety impairment in Alzheimer's disease.

Thus, s-KL for use according to the invention is, in a particular embodiment for the prevention and/or treatment of cognitive and/or behaviour impairment in mammals, and particularly in humans. Klotho protein has a high percentage of homology among mammals as it is shown in the following table (analysis by BLAST, amino acid sequences from NCBI):

| | Rat | Dog | Mouse | Goat | Cebus |
|---|---|---|---|---|---|
| Human | 92% | 94% | 92% | 93% | 93% |
| Cebus Capuccino | 90% | 92% | 90% | 92% | — |
| (primate) | | | | | |
| Goat | 93% | 94% | 93% | — | — |
| Mouse | 97% | 93% | — | — | — |
| Dog | 92% | — | — | — | — |
| Rat | — | — | — | — | — |

In a particular embodiment, s-KL for use as above exposed is a polypeptide selected from SEQ ID NO: 1, SEQ ID NO: 2 and a polypeptide with a percentage of identity of at least 88% with any of SEQ ID NO: 1 or SEQ ID NO: 2. The percentage of identity determined by using the BLASTP algorithm.

In yet a more particular embodiment it is a polypeptide selected from amino acid sequences SEQ ID NO: 1 and SEQ ID NO: 2. In other particular embodiments, s-KL for use as above exposed is a polypeptide with a percentage of identity with either SEQ ID NO: 1 or SEQ ID NO: 1 from 88% to 100%. Ranges of identity percentages comprise 88%, 88.5%, 89%, 89.5%, 90%, 90.5%, 91%, 91.5%, 92%, 92.5%, 93%, 93.5%, 94%, 94.5%, 95%, 95.5%, 96%, 96.5%, 97%, 97.5%, 98%, 98.5%, 99%, 99.5% and 100%. In another more particular embodiment the percentage of identity of the polypeptide is from 88% to 90%; and yet more particularly from 88% to 88.5%.

SEQ ID NO: 1 is the amino acid sequence of the transcript from alternative splicing of a-klotho human gene, comprising the KL1 domain sequence, with an approximate weight of 70 kDa, but with a specific secretion signal consisting of 15 amino acid tail that is not found in the m-KL transcript, a-klotho human gene is the one located in Chromosome 13 NC_000013.11 (33016063 . . . 33066145) of the assembly GRCh38 (24.12.2013) for the human genome maintained by the Genome Reference Consortium. SEQ ID NO: 1 derives from the corresponding cDNA of SEQ ID NO: 5, deriving from the alternative splicing transcript of the mRNA sequence with the GenBank database accession number NM_004795 of 5012 base pairs, version 3 of 3. May.2014.

SEQ ID NO: 2 is the amino acid sequence of the transcript from alternative splicing of a-klotho mouse gene, comprising the KL1 domain sequence, with an approximate weight of 70 kDa, but with a specific secretion signal consisting of 15 amino acid tail that is not found in the m-KL transcript, a-klotho mouse gene is the one located in Chromosome 5 (150,952,607-150,993,809) of UCSC Genome Browser on Mouse July 2007 (NCBI37/mm9) Assembly for the mouse genome. SEQ ID NO: 2 derived from the corresponding cDNA of SEQ ID NO: 6, deriving in turn from the alternative splicing transcript of the mRNA sequence with the GenBank database accession number NM_013823 of 5124 base pairs, version 2 of 15.February.2015.

The polypeptides with a percentage of identity of at least 88% with any of SEQ ID NO: 1 or SEQ ID NO: 2 encompass mammal proteins derived from amino acid variations of the sequences including single or of two or three amino acid substitutions in SEQ ID NO: 1 or 2, deletion of one or two amino acids, insertion of one or two amino acids at any position of the sequence, all these amino acid variations in relation to SEQ ID NO: 1 or 2 with the proviso that the resulting proteins have the same function as the s-KL from which derive. The polypeptides with a percentage of identity of at least 88% with any of SEQ ID NO: 1 or SEQ ID NO: 2 encompass also s-KL of mammals other that mice and human. As above indicated, the identity is determined by global alignment between sequences performed by means of the BLASTP algorithm.

In another particular embodiment of the first aspect of the invention, the s-KL for use as above exposed is a polypeptide consisting in SEQ ID NO: 1 or SEQ ID NO: 2.

s-KL may be used directly in the form of the protein, conveniently directed or finally reaching brain or central nervous system (CNS). This protein can be administered, for example in the form of a pharmaceutically composition comprising a therapeutically amount of the protein suspended o dissolved in a carrier (solvent) useful for injection into the brain or useful for intravenous injection (mainly), together with acceptable excipients for stabilizing the protein.

On the other hand, s-KL can be expressed inside target cells of CNS by means of gene therapy. To this aim the invention also provides the new gene construct comprising a nucleic acid sequence coding for the secreted splicing variant of mammal klotho protein (s-KL) operatively linked to an expression promoter. When in the present invention it is said “a nucleic acid sequence coding for the secreted splicing variant of mammal klotho protein (s-KL)” is to be understood, in particular, the cDNA sequence resulting from the reverse transcription (RT-PCR) of mRNA for said s-KL in the mammal. In a particular embodiment the expression promoter operatively linked is selected from a constitutive expression promoter, an inducible promoter and a neuron-specific expression promoter. In a particular embodiment, the gene construct according to the invention comprises the nucleic acid sequence SEQ ID NO: 3. SEQ ID NO: 3 comprises the cytomegalovirus intermediate-early (CMV IE) promoter, the sequence coding for s-KL (cDNA of mouse s-KL) and a polyadenylation chain (poly A). In another particular embodiment the gene construct according to the invention comprises the nucleic acid sequence SEQ ID NO: 4, equivalent to SEQ ID NO: 3 but with the sequence coding for human s-KL protein (cDNA of human s-KL).

In another particular embodiment the gene constructs consist in either SEQ ID NO: 3 or SEQ ID NO: 4.

All these gene constructs are able to express the protein of interest once in the cell, and particularly in the CNS cells.

In order to facilitate administration of the constructs the invention also proposes new expression vectors with central nervous system tropism comprising the gene construct as defined above, thus comprising a nucleic acid sequence coding for the secreted splicing variant of mammal klotho protein (s-KL) operatively linked to an expression promoter, and particularly to a constitutive expression promoter.

Suitable expression vectors for the purposes of the invention are vectors with central nervous system (CNS) tropism or that effectively transduce CNS cells. Expression vectors for gene therapy are usually viruses, and particularly retroviruses, adenoviruses, envelope protein pseudotyping of viral vectors, replication-competent vectors, cis and trans-acting elements, or Herpes simplex virus.

In a particular embodiment of these expression vectors, they are viral vectors. More particularly, the expression vectors are adeno-associated virus, and more particularly adeno-associated virus with CNS tropism, which can be of serotype 1-10. Particularly, the adeno-associated virus is of serotype rh 10 (AA VrhI O).

Other possible vectors for the delivery of the gene constructs comprising s-KL coding sequence include liposomes, micro-and nanoparticles that avoid damage of the gene construct and moreover facilitate entrance of the construct inside the CNS cells in an effective and specific manner. s-KL in the form of plasmids or naked DNA can also be administered for gene therapy by non-viral methods, such as injection of naked DNA, physical methods to enhance delivery, electroporation, gene gun, sonoporation, magnetofection, or hydrodynamic delivery. Other chemical methods to enhance delivery are the use of oligonucleotides, lipoplexes, polymersomes, polyplexes, dendrimers or inorganic nanoparticles.

Another aspect of the invention is, as above exposed, a combination of the secreted splicing variant of mammal klotho protein and/or the nucleic acid construct as defined above, and/or the vector as defined above with another active agent for use in the prevention and/or treatment of cognitive and/or behaviour impairment associated with aging, and/or with neurodegenerative and/or neuropathological diseases.

In a particular embodiment, said combination is for the prevention and/or treatment of cognitive and/or behaviour impairment associated with Alzheimer's disease (AD). The combination relates to a pharmaceutically composition or preparation for administration of s-KL with any other active agent, such as donepezile hydrochloride (Aricept, Pfizer), memantine, rivastigmine, and ligustilide, which can be done separately, in any order, within a therapeutically effective interval, or for the simultaneously administration of the active agents. In yet a more particular embodiment the combination is for use in the prevention and/or treatment of anxiety in patients of AD.

s-KL can be administered to the patient via mucosa (e.g., nasal, sublingual, vaginal, buccal, or rectal), parenterally (e.g., subcutaneous, intravenous, intramuscular, or intraarterial injection, either bolus or infusion), orally, transdermal\hat{ } or via inhalation by means e.g. of an aerosol. Formulations suitable for parenteral administration, such as, for example, by intraarticular, intravenous, intramuscular, intradermal, intraperitoneal, and subcutaneous routes, include aqueous and non-aqueous, isotonic sterile injection solutions, which can contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. Injection solutions and suspensions can also be prepared from sterile powders, granules, and tablets. In some embodiments, the composition is administered by injection e.g subcutaneous, intraperitoneal, intravesically, intravenous, by infusion, e.g., using a reservoir or osmotic minipump or intramuscular. The formulation can be provided in unit-dose or multi-dose sealed containers, such as ampoules and vials.

In a particular embodiment s-KL is administered to the patient parenterally and more particular, intravenously.

In other particular embodiments s-KL is administered with direct deliver to the central nervous system, more particularly injected intrathecally or intracisterna magna (ICM), and particularly by means of a patch, a micropump or a microcapsule delivery system.

Throughout the description and claims the word “comprise” and variations of the word, are not intended to exclude other technical features, additives, components, or steps. Furthermore, the word “comprise” encompasses the case of “consisting of. Additional objects, advantages and features of the invention will become apparent to those skilled in the art upon examination of the description or may be learned by practice of the invention. The following examples are provided by way of illustration, and they are not intended to be limiting of the present invention. Furthermore, the present invention covers all possible combinations of particular and preferred embodiments described herein.

### EXAMPLES

### Example 1. Generation of Plasmidic Vectors Coding for Secreted Klotho Protein, Production of AAV Viral Vectors for Transduction

Generation of viral vectors for gene therapy was performed from synthetic constructs. Gene construct (plasmid) pCR-KL-TOPO-KL (ImaGenes™, Germany) coded for full-length cDNA of the transmembrane protein isoform of Klotho (m-KL). Klotho sequences were then cloned in pGG2 plasmid (of SEQ ID NO: 12 and courtesy of Genethon) by means of ligation with restriction enzymes, said pGGs plasmid carrying the Inverted Terminal Repeat (ITR) sequences of the adeno-associated virus serotype 2 (AAV2) genome and a multiple cloning site (MCS) where the gene of interest was cloned under the control of cytomegalovirus immediate-early promoter (CMV IE). To do this a cDNA of m-KL was extracted from pCR-KL-TOPO-KL with Xbal/EcoRI restriction enzymes and cloning the fragment in vector p123T comprising sequence of CMV IE promoter (MoBiTec, Alemania), EMBL-EBI accession number Z46733, release 121 of 29.Aug2014) using the same enzymes. The fragment comprising sequences of CMV IE promoter and the sequence coding for m-KL (herewith termed CMV 1E-mKL) was cloned in pGG2 plasmid, comprising sequence of CMV IE promoter, to obtain the expression cassette flanked with Inverted terminal repeats (ITR). This plasmid was termed pGG2-m-KL (of SEQ ID NO: 13).

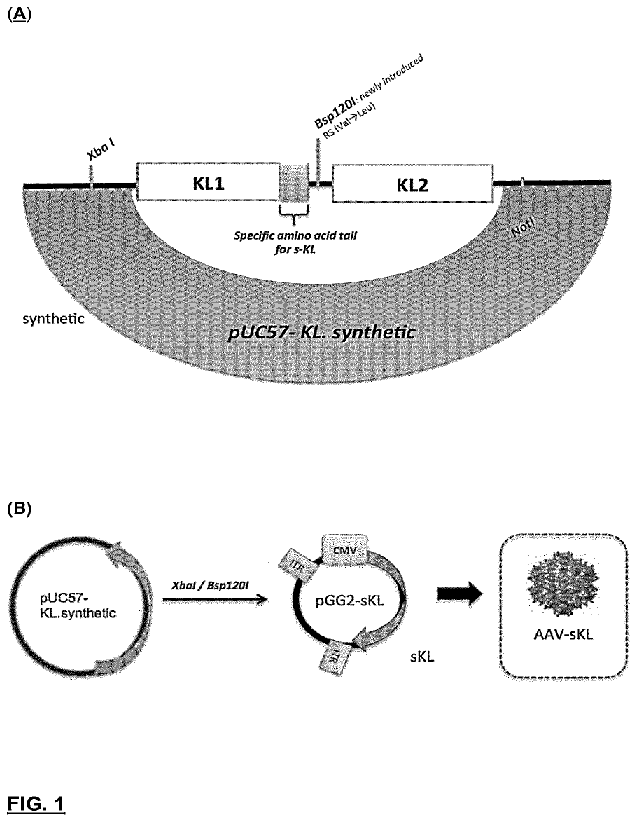

A gene construct comprising the cDNA coding for s-KL was generated from synthetic pUC57-KL. (GenScript, USA) of SEQ ID NO: 14. This plasmid contained appropriate restriction sites for cloning s-KL. In FIG. 1 (A) it is schematically depicted the plasmid map. FIG. 1 (B) shows strategy to clone the s-KL isoforms that could be derived from synthetic pUC57-KL.

A plasmid including the s-KL coding sequence (pGG2-s-KL, SEQ ID NO: 15) was generated with Xbal/Bsp1201 targets, which are compatible with Notl/Xbal to open pGG2 vector. pGG2-s-KL and pGG2-m-KL were in this particular example obtained with pGG2 plasmid. Anyway, other plasmids are useful while allowing packaging in viral capsid of the gene constructs comprising CMV IE promoter operatively linked to the sequence coding for s-KL and a polyadenylation chain (SEQ ID NO: 3 for murine s-KL, and SEQ ID NO: 4 for human s-KL); as well as of the same constructs for m-KL (human or murine). The packaging of these sequences leads to the same viral genome and viral capsid proteins, independently of the plasmids used for the generation of AAV as exemplified below.

Further AAV vectors comprising either plasmid pGG2-mKL or pGG2-sKL were generated by means of triple transfection of 293-AAV cells from Stratagene (70% confluency) with the pGG2 plasmids, the pXX6 plasmid (SEQ ID NO: 16, by courtesy of Gencthon), carrying AAV genes for AAV amplification, and the plasmid pREp2Cap10 (SEQ ID NO: 17) (MTA Dr. J. M. Wilson, University of Pennsylvania), this later carrying sequences Cap and Rep of AAVrhl O. Viruses were then purified using ultra centrifugation in iodixanol gradient, counted by picogreen method (Piedra et al., “Development of a rapid, robust, and universal picogreen-based method to titer adeno-associated vectors”, Hum Gene Ther Methods—2015, vol 26 (1), pp: 35-42; or doi: 10.1089/hgtb.2014.120 PMID: 25640021) and stored at-80° C. until moment of use. The expression vectors were named AAVrh10_pGG2-sKL and AAVrh 10_pGG2_mKL. Primers for quantification of AAVrh 10_pGG2_mKL are m-KL-forward 5′-TTCAAACCCGGAAGTCTTTG-3′ (SEQ ID NO: 7), and m-KL-reverse 5′-CCAGGCAGACGTTCACATTA-3′ (SEQ ID NO: 8).

The procedure was as follows: 20 plates of 15 cm of diameter with 293-AAV cells from Stratagene (70% confluency) were transfected with Polyethyleneimine PEI (PolyScience) with 500 g of pXX6, 250 g of pRep2Cap10 and 250 g of pGG2 plasmid. The three plasmids were mixed in media DMEM and added to the plates to a final volume of 14 ml/plate. 6 hours later the media was changed. 48 hours post-transfection cells were scrapped and centrifuged. They were resuspended in lysis buffer (50 mM Tris (Sigma), 20 mM NaCl (Panreac) and 2 mM MgCI2(Panreac)). For AAV purification and after 3 cycles of frost and defrost, cell residues were obtained by centrifugation and supernatant was saved. Benzonase (50 U/ml) was added to the supernatant to decompose cell DNA. Viral particles were further precipitated with polyethyleneglycol (PEG at 1 ml/4 ml of cell lysate). Centrifugation at 8000 g for 15 minutes allowed a pellet with the viral particles. 15 ml of lysis buffer were added to iodixanol gradient tubes and the viral particles removed after centrifugation at 690000 g for 1 hour.

### Example 2. In Vivo Administration of AAV s-KLvectors in Old Mice

### Example 2A: Administration of AAV Vectors

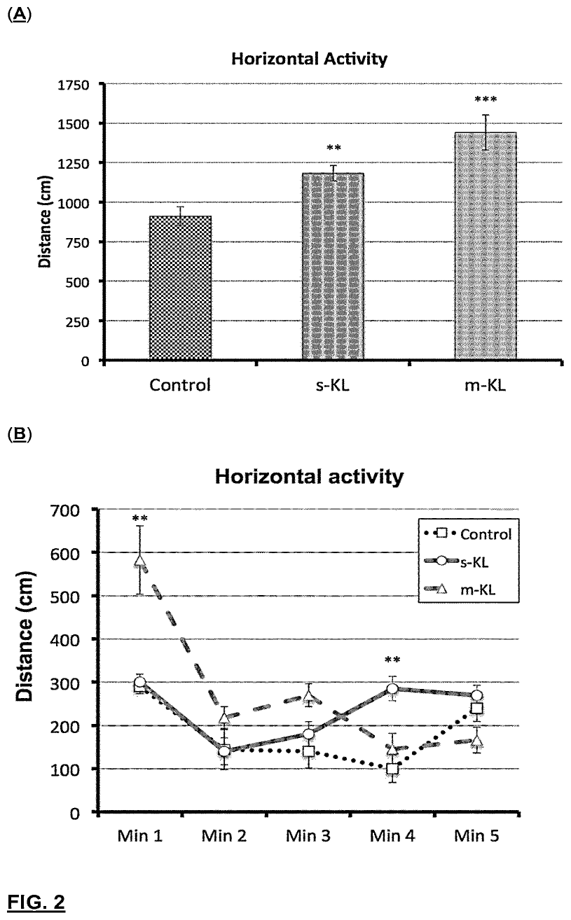

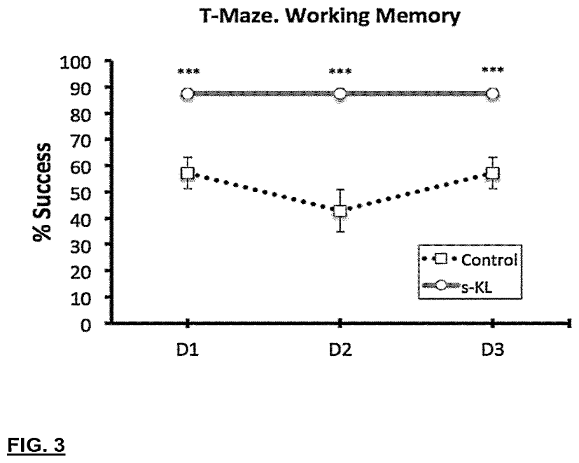

Long-term effects of klotho overexpression in the aging CNS were evaluated in C57BL/6 mice injected at 12 months of age (middle-aged, N=10), and tested 6 months later, when they reached old age (18 months), through a battery of tests for behavioural assessment and functional analysis (FIG. 2 and FIG. 3). The control group was injected with an AAVrhI O vector encoding an irrelevant DNA sequence and the treated group with an AAVrhI O vector encoding the secreted (s-KL) Klotho isoform. AAV vectors were injected intracerebroventricularly (icv) to mimic the endogenous production system, in which Klotho produced in the CNS is released into the CSF and distributed throughout the brain.

For the in vivo assays, C57B16 mice of Harlan Laboratories BV (wild-type males) were used. All experiments with animals and necropsies were done in Universitat Autonoma de Barcelona (Spain) according to Spanish applicable law. Protocols were approved by Ethics Committee. Animals were fed ad libitum in Macrolon cages at 22° C. and 12 hours of dark and light cycles starting at 8:00 h.

Mice were anesthetized by intraperitoneal injection of ketamine (10 mg/kg of body weight; Imalgene 500; Rhone-Merieux) and xylazine (1 mg/kg of body weight; Rompun; Bayer) and mounted onto a stereotactic frame (David Kopf Instruments, CA, USA). Wild-type mice (12 months old) were injected in the brain to reach cerebrospinal fluid (CSF) with the expression vectors of Example 1. So, there were generated s-KL mice (injected with AAVrh10_pGG2-sKL, also abbreviated AAVrh10-s-KL). As control (Null mice), a vector coding for an irrelevant gene was used. Experimental groups consisted in 10 animals per group (n=10). Intraventricular injection was performed in the third lateral ventricle of brain with the aid of a stereotaxic device. The administered dose of the expression AAV vector was of 1.1010 vector genomes per mouse (vg/mouse) in a single dose of 3 μt.

### Example 2B. Overexpression of s-KL in the CNS Does not Affect Body Weight or Sensorimotor Skills in old Mice

Behaviour and cognitive tests were conducted when mice were 18-months old. Firstly, sensorimotor tests were conducted (Day 1), followed by the corner test and the open field test (Day 2). Next, it was performed the T-maze test (Day 3-5). The Morris Water Maze (MWM) test was done at the end (Days 6-12).

The body weight of animals was monitored once a month, from 12 to 19 months of age. Reflexes (visual reflex and posterior legs extension reflex tests) were measured three times by holding the animal by his tail and slowly lowering it onto a black surface. The motor coordination and equilibrium were assessed by the distance covered and the latency to fall off a horizontal wooden rod (1.3 cm wide) on two consecutive 20 s trials, respectively. In order to increase the difficulty of the task, the test was repeated on a metal wire rod (1 cm diameter). Prehensility and motor coordination were measured as the distance covered on the wire hang test, which consisted in allowing the animal to cling from the middle of a horizontal wire (diameter: 2 mm, length: 40 cm, divided into eight 5 cm segments) with its forepaws for two trials of 5s and a third 60 s trial. Muscle strength was measured as the time until falling off the wire in the 60 s trial. All the apparatus was suspended 40 cm above a padded table.

Behavioural assessment of treated animals started with the evaluation of putative effects of s-KL in physical status, namely body weight and reflexes as well as basic sensorimotor functions such as balance, coordination, prehensility, strength and resistance. These measures allow detecting possible differences between groups that could subsequently affect results depending on motor performance. Of note, there were no significant differences in the mean weight between both groups of animals (p=0.48). However, s-KL treated mice had a relatively stable body weight throughout the 6 months post injection, whilst the null-treated group's weight progressively increased during middle age and decreased in old age. The results shown in

Table 1, also show a similar sensorimotor function in s-KL treated and control groups as measured by the visual placing and hindlimb reflexes test, the wood and metal rod tests; and the hanger tests. It is therefore assumed that all animals were in similar physical conditions when analyzed in subsequent behavioural tests.

TABLE 1
Sensorimotor Tests

| | Control | s-KL |
|---|---|---|
| Visual reflex (correct) | 100% | 100% |
| Wooden Bar (s) | 19.0 ± 1.0 | 17.86 ± 1.25 |
| Metal Bar (s) | 3.5 ± 0.67 | 3.21 ± 1.19 |

### Example 2C: s-KL Overexpression in the CNS Ameliorates age-related Motor Decline Without Affecting Anxiety-like Behaviours in old Mice

Human aging, both normal and in age-related diseases, is associated with highly correlated changes in motor performance that parallel alterations in cognition and motivation. Therefore, since locomotion and exploratory behaviours decrease with age, we first sought to determine whether overexpression of the secreted Klotho isoform in the CNS could affect locomotor/exploratory behaviour in injected mice compared to controls using the Open Field Test.

This test was developed to study neophobia and anxiety-like behaviours and is most often used in rodents to qualitatively and quantitatively measure general locomotor activity (horizontal and vertical activities) and willingness to explore (mostly shown by the vertical activity). Open field activity, including total distance travelled, rearing exploratory behaviour, latency of behavioural events, self-grooming behaviour and defecations, were examined in order to determine whether sKL overexpression in the aged mice brain elicited changes in locomotion, exploratory activity, emotional and anxiety-like behaviors.

In order to know specifically whether m-KL and s-KL may induce different effects in the treated animals, wild-type mice (12 months old) were also injected intraventricularly in the brain with the AAVrh10_pGG2_mKL vector (n=10).

Results of this test are depicted in FIG. 2 (A, B). In FIG. 2(A) total distance (cm) made in open field is depicted in bars. In FIG. 2(B) there are recorded the total distance (cm) made per minute for each type of mouse. In Table 2 different parameters to evaluate the exploratory activity of the animals in the open field test. Table 2: Exploratory activity in the open field test.

TABLE 2
Exploratory activity in the open field test

| | Control | s-KL |
|---|---|---|
| Latency to leave the center(s) | 6.75 ± 1.79 | 3.98 ± 1.11 |
| Latency to arrive to periphery (s) | 9.14 ± 1.36 | 7.7 ± 1.09 |
| Latency first rearing (s) | 18.42 ± 2.91 | 24.33 ± 3.92 |
| Number of groomings | 2.86 ± 0.10 | 2.29 ± 0.12** |
| Latency first grooming (s) | 84.17 ± 11.63 | 90.33 ± 24.06 |
| Number of defecations | 2.13 ± 0.49 | 1.94 ± 0.32 |

As shown in FIG. 2A when tested at the age of 18-19 months, mice previously icv administered at 12 months with s-KL or m-KL travelled a greater total distance compared to control mice (p<0.01; p<0.001 respectively). Interestingly, the greater locomotor activity observed in m-KL treated mice appears to be related to anxiety, since differences in the distance travelled were not observed in the first three minutes of the test (FIG. 2B). In comparison, the greater locomotor activity observed for the s-KL mice is not associated with anxiety since the distance run is statistically significant at the end of the test, and therefore is more associated with exploratory behaviour, which is more typical of younger animals (FIG. 2B). This is in agreement with the other parameters measured, such as the sequence of behavioural events and the number of defecations, which indicate similar levels of neophobia and emotionality in both groups of animals when confronting the open arena (Table 2). Only, a reduction in the number of groomings (p=0.0032) in treated animals was statistically significant.

### Example 2D: CNS s-KL Overexpression Improves Cognitive Performance in old Mice

In order to study the possible long-lasting neuroprotective effect of Klotho in the CNS, animal's cognitive skills were evaluated in two learning and memory tests: T-maze and Morris Water Maze.

The spontaneous exploratory behavior was tested in a T-shaped maze (arms, length 25 cm). Animals were placed inside the vertical arm of the maze facing the end wall. The performance was evaluated by determining the time elapsed until the animal crossed (four-paw criteria) the intersection of the three arms. The working memory paradigm consisted in two consecutive trials: one forced choice and one free choice, with a 90 s intertrial interval. In the forced choice, only one of the arms according to a random order (contrabalanced in each group) was accessible. Each mouse was placed in the “vertical” arm of the maze with its head facing the end wall and it was allowed to explore the maze. After spending 20s in the accessible arm (learning criterion), the animal was put back into the home cage starting box. Nineteen seconds later, it was again allowed to explore the maze in a free choice trial where both arms were accessible. The arm chosen by the mouse and the time spent in each arm during the free choice was recorded. The choice of the already visited arm in the previous trial before exploring the arm that was inaccessible was considered as an error. Also the time spent to complete the exploration of the three arms in the maze was recorded. The olfactory trails were removed by cleaning the surface of the maze during the intertrial intervals.

TABLE 3
Different parameters in the forced choice and free-choice trial

| | |
|---|---|
| Control | s-KL |

Forced Choice in T-maze

| | | |
|---|---|---|
| T-intersection (latency, s) | 15.14 ± 1.38 | 12.04 ± 1.11 |
| Criteria Achievement (frequency/total n) | 6/8 | 5/6 |
| Exploration criteria (latency, s) | 76.19 ± 3.13 | 102.0 ± 14.56 |

Memory Assay in T-maze

| | | |
|---|---|---|
| Criteria achievement (frequency/total n) | 5/6 | 5/5 |
| Errors (frequency/total n) | 2.94/6 | 0.65/5** |

As shown in Table 3, in the forced choice tasks in the T-maze all groups met the criteria similarly, although not all animals were able to complete the task. Subsequently, only those animals that met the criteria were administered a second trial, the memory task. Results show that the increase in s-KL expression in the CNS in aged mice significantly improved their memory score (frequency of errors of 0.65/5) compared to the control group (frequency of errors of 2.94/6). This is demonstrated by significantly fewer errors in choosing the maze path (p=0.0018). Moreover, this ability was sustained over three consecutive days (FIG. 3). These data indicated that an increase in the expression of s-KL in CNS allows improving punctuation in relation to controls. This moreover implies a better working memory, said memory of particular interest in aging people and in Alzheimer's disease.

Further analysis of cognitive abilities in treated animals was performed in the Morris Water Maze. This test consists of one cue task for visual perceptual learning, four days of place task for spatial reference learning and memory followed by a probe trial for long-term (24 h) memory. In the place-learning task, mice were trained to locate a platform (7 cm diameter, 1.5 cm below the water surface, position indicated by a visible 5×8 cm striped flag) in a circular pool (Intex Recreation Corp. CA, USA; 91 cm diameter, 40 cm height, 25°° C. opaque water) located in a test room with distal visual cues. This required four platform trial sessions per day with trials spaced 15 min apart. In each trial, the mouse was gently released (facing the wall) from one randomly selected starting point (N, S, E, or W) and allowed to swim until escaping onto the platform (always in the middle of the SE quadrant). Mice that failed to find the platform within 60 s were placed on it for 20 s, the same period as was allowed for the successful animals. Twenty-four hours after the last cued platform trial, animals were tested for the cue learning of a visual platform consisting of four hidden platform trials (20 min apart). The platform was hidden 1.5 cm below the water surface, with its new position (NW) indicated by a visible striped flag (5\hat{ }8 cm), and the distal cues were removed. During each trial, the escape latency, the distance traveled, and the mean speed were measured by means of a computerized tracking system (SMART, Panlab S.A., Spain).

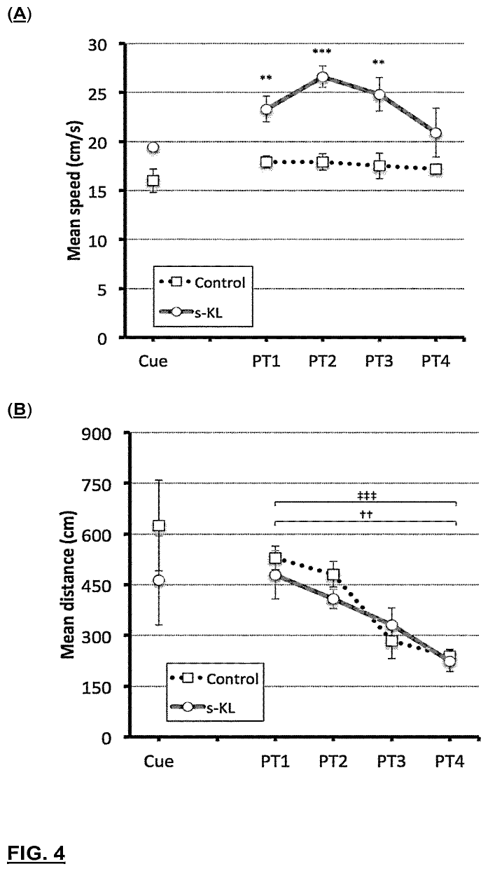

Notably, swimming speeds were significantly higher in the group treated with s-KL (FIG. 4A). This is in agreement with the increased horizontal locomotor activity observed in these same animals in the open-field test. We therefore considered distance was a more accurate variable than latencies or speed to evaluate learning and memory skills in these animals. Importantly, despite their age, both groups demonstrated learning over the four training days since there was a gradual reduction in the distance covered to solve the task (FIG. 4B, p<0.01 for Control; p<0.001 for s-KL). Finally, the acquisition of spatial learning was similar, regardless of the group. This indicates that before the memory tests, all animals were equal when facing the task.

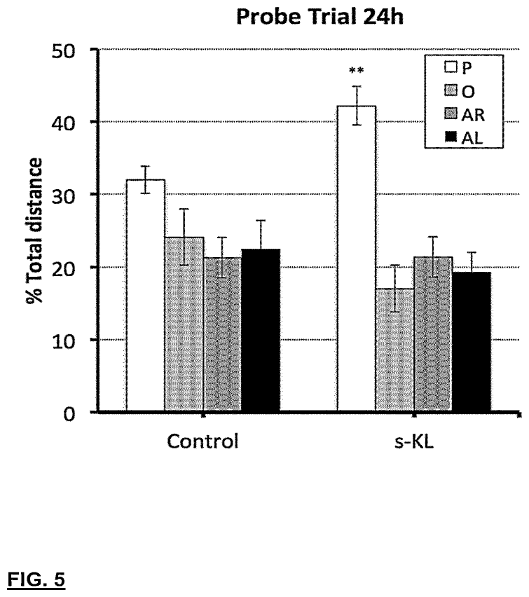

Finally, 24 hours after the last session of acquisition (Probe Trial 24h), aged control mice had a poorer performance in the long-term memory trial. They showed less preference for the training quadrant than they displayed in the short-term memory trial. In contrast, the s-KL treated group had a clear preference for the training quadrant, statistically significant from the control (p<0.01, FIG. 5). These results indicate that the cognitive effects of increased levels of s-KL in the CNS are connected with selective improvement in long-term memory.

### Example 2E. Quantification of Viral Genomes in the CNS of Injected Mice

After all tests were terminated mice were sacrificed and one half of brains fixed in paraformaldehyde (4%) for histology. Other half part of brains was used for determining in different brain sections (prefrontal cortex, PFC; cortex, C; hippocampus, H; and cerebellum, CB), the presence of viral genomes (vg) and the expression levels of s-KL.

For these analyses, quantitative PCRs were performed. For the detection of viral genomes the DNA Hirt extraction process (Hirt B et al., “Selective extraction of polyoma DNA from infected mouse cell cultures”, J Mol Biol-1967, vol 26, pp: 365-369) was conducted, allowing isolation of viral episomes from the inside cells. Quantitative PCR was carried out with specific primers for CMV IE promoter. Primers used to detect by quantitative PCR (qPCR) levels of s-KL were:

s-KL-forward
(SEQ ID NO: 9)
5′-TGGCTTTCCTCCTTTACCTG-3′,
s-KL-reverse
(SEQ ID NO: 10)
5′-GCCGACACTGGGTTTTGT-3′,
CMV-Fwd:
(SEQ ID NO: 18)
5′-TACATAACTTACGGTAAATGGC-3′
and,
CMV-Rev:
(SEQ ID NO: 19)
5′-AAAGTCCCTATTGGCGTTACT-3′.

Viral expression was conducted by extracting RNA from the brain sections. qPCR was carried out following suppliers instructions with iTaq Universal SYBR Green Supermix (BioRad), the thermocycler CFX384 TouchTM Real-Time PCR Detection System (BioRad), Hard-Shell Rhin-Wall 384-Weel Skirted PCR Plates (BioRad), Microseal “B” Adhesive Seals (BioRad). cDNA was diluted ⅕. Amplification program was of 98° C. for 2 minutes, 40 cycles at 95° C. for 5 seconds and 58° C. for 30 seconds.

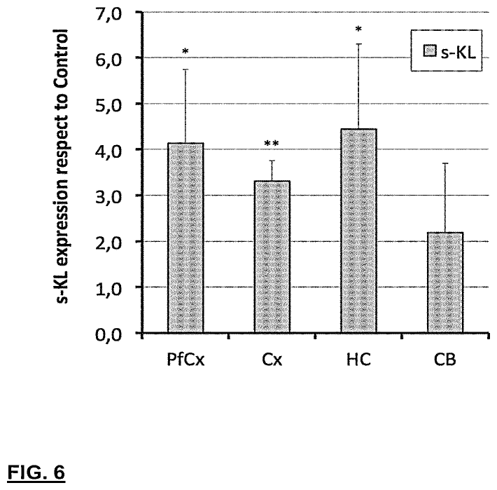

AAV genomes were detected in all injected animals, AAV-s-KL distributed similarly to AAV-Null control (data not shown). Thus 6 months after intraventricular administration, AAVrhI O vector was still present in the CNS of 18 month-old mice. More importantly, s-KL expression was increased in all the brain areas analyzed (by qPCR on s-KL mRNA), ranging from 2 times higher in cerebellum to 4 times in prefrontal cortex and hippocampus (FIG. 6). These results confirm that the differences in the behaviour and cognitive tests disclosed above for old mice where due to s-klotho over-expression.

### Example 3. In vivo Administration of s-KL in Young Mice

In order to study whether long-term expression of s-KL could improve the physical, non-cognitive and cognitive status of middle-aged animals, a second set of mice (n=11-14) was treated at 6 months of age. They were then assessed at 12 months by the same battery of tests. There is a much lower efficiency of AAV transduction in older compared to younger brains. By testing a group of relatively younger adult animals, we could avoid this limitation. In addition, for this group AAV was specifically injected into the hippocampus. The main reasons were (i) the klotho gene is abundantly expressed in hippocampus; (ii) hippocampus is involved in learning and memory processes; and (iii) in mice, the hippocampus develops structurally until 12 months of age, later undergoing age-dependent functional decline. Actually, there are other brain regions where klotho is also highly expressed (e.g. plexus choroide, and cerebellum). However, we were interested to see if specifically enhancing s-KL in the hippocampus during a period of plasticity, would improve hippocampal-dependent learning and memory processes and thereby reduce the future impact of functional decline.

In the same experimental design, we also compared whether s-KL inhibition worsened cognitive deficits in naive mice, with respect to control-treated and s-KL treated animals. To achieve specific inhibition of the secreted Klotho isoform, we administered AAV vectors carrying shRNA against s-KL. The shRNA sequence was designed against the extra sequence in the tail of s-KL not present in m-KL. An AAV carrying a shRNA-scrambled sequence was used as a control.

### Example 3A

Administration of AA Vectors In vivo Assay was performed as in Example 2, but with young animals (6-months old when injected with the AAV vectors comprising coding sequences for s-KL, or scramble DNA as control). In this case animals were injected in hippocampus (2 injections of 5×109 vg/mouse, one injection per hemisphere). Animal body weight was measured at the beginning of the experiment (6 months), at 9 and 12 months of age. The same behaviour and cognitive tests were conducted when mice were 12-months old. As in Example 2, weight (monthly determined) was increased during the assay, as expected. Of note, sustained overexpression or inhibition of s-KL over time had no significant effect and all groups showed a steady, similar weight gain (data not shown).

TABLE 4
Sensorimotor functions

| | Null | s-KL |
|---|---|---|
| Visual reflex | 3/3 | 3/3 |
| (Extension of extremities when mice are | | |
| approximated to a black panel while being | | |
| hanged through the tail. If done | | |
| punctuation of “1” is accorded) | | |
| Wooden bar | 14.45 ± 1.95 | 17.92 ± 1.16 |
| (Latency to fall down, in seconds, of | | |
| a 40 cm high suspended bar) | | |
| Metallic bar | 9.09 ± 1.86 | 6.85 ± 1.27 |
| (Latency to fall down, in seconds, of | | |
| a 40 cm high suspended bar) | | |
| Hanger | 34.36 ± 7.04 | 39.57 ± 5.84 |
| (Latency to fall down, in seconds, of | | |
| a 60 cm high suspended hanger, where | | |
| mice are hanged by means of frontal | | |
| extremities) | | |

Likewise in the previous experiment with older animals, sustained overexpression or inhibition of s-KL from 6 to 12 months of age did not affect the reflexes and sensorimotor skills of all groups, the reflexes and sensorimotor skills of all groups were not affected (Table 4).

### Example 3B

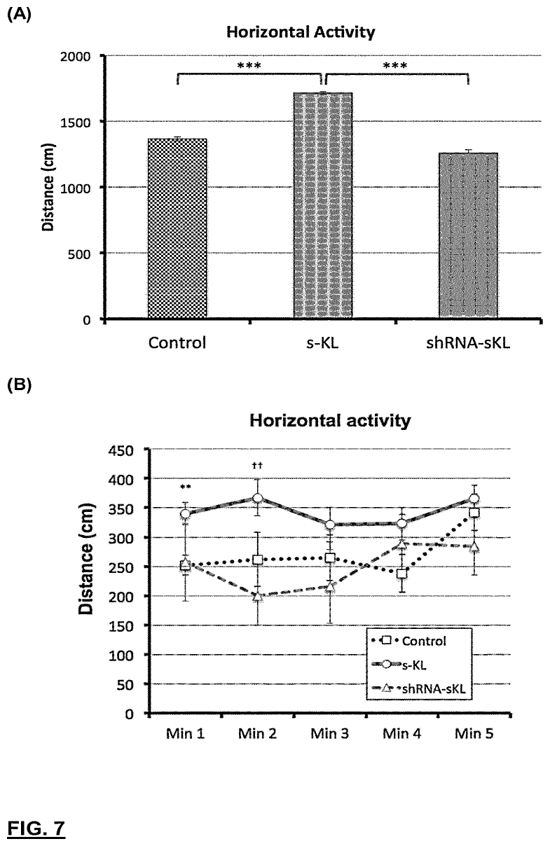

Hippocampal s-KL overexpression results in an increase in horizontal activity and has a mild anxiolytic effect in middle-aged adult mice. In the open field test (FIG. 7) it could also be observed that over-expression of klotho induced changes in spontaneous activity in relation to wild-type mice. Consistent with previous results in aged mice, modification of s-KL levels by gene therapy approaches was able to change horizontal locomotor activity (FIG. 7A, 7B), but not the vertical activity (data not shown) of mice in the open field test. Thus, when s-KL cDNA was overexpressed in hippocampus, the total distance travelled by mice was significantly higher than in the other two groups (p<0.0001) (Control: 1365+14 cm; s-KL: 1734+8 cm; shRNA/s-KL: 1259 +27. cm). On the other hand, during the first minute of the test, some control animals froze for a while which is a direct measure of increased anxiety. In the case of mice overexpressing s-KL, a shorter latency to leave the central area was recorded although this difference was only statistically significant with respect to shRNA-sKL injected mice (p=0.0008) (Table 5). No differences were detected between the groups in the other variables analysed.

TABLE 5
Exploratory activity in the open field test

| | Control | s-KL | shRNA-sKL |
|---|---|---|---|
| Latency to | 5.18 ± 1.60 | 3.00 ± 0.97*** | 8.00 ± 0.78 |
| leave the | | | |
| center(s) | | | |
| Latency to | 9.12 ± 1.57 | 7.33 ± 1.22 | 10.34 ± 1.09 |
| arrive to | | | |
| periphery (s) | | | |
| Latency first | 27.63 ± 3.96 | 26.92 ± 4.08 | 23.80 ± 2.74 |
| rearing (s) | | | |
| Number of | 1.81 ± 0.42 | 1.42 ± 0.32 | 1.81 ± 0.31 |
| groomings | | | |
| Latency first | 115.54 ± 25.49 | 164.92 ± 23.16 | 112.36 ± 18.43 |
| grooming (s) | | | |
| Number of | 2.33 ± 0.47 | 1.96 ± 0.35 | 2.4 ± 0.40 |
| defecations | | | |

### Example 3C. Hippocampal s-KL Overexpression Improves Cognitive Performance in Middle-aged Adult Mice, While s-KL Inhibition Impairs it

When working memory is evaluated in T Maze test, also meaningful differences are observed between treated and control animals.

Results are presented as the mean values obtained for each of the three test days. First, in the forced-choice trial, all control animals and those overexpressing s-KL were able to meet the criteria. On the contrary, about 10% of shRNA-sKL treated animals did not, and therefore were discarded. In addition, in the first trial, mice overexpressing s-KL needed less time to reach the intersection point of the maze compared to mice injected with shRNA-sKL (p=0.009) (Table 6). Thereafter, in the free-choice trial, all animals met all the established criteria, but differences between groups were observed in terms of efficiency in choosing the correct path. The control group solved the task with an error rate of 36.36%. This score was improved in the s-KL overexpression group with an error rate of 21.42%. In contrast silencing s-KL increased the percentage of error up to 40% (Table 6).

TABLE 6
Different parameters in the forced choice and free-choice trial of a T-
maze test performed in control, s-KL, and shRNA-sKL mice

| | | |
|---|---|---|
| Control | s-KL | shRNA-sKL |

Forced choice in T-maze

| | | | |
|---|---|---|---|
| T-intersection (latency, s) | 8.48 ± 1.77 | 5.29 ± 1.29** | 10.28 ± 1.11 |
| Criteria Achievement | 11/11 | 14/14 | 10/11 |
| (frequency/total n) | | | |
| Exploration criteria | 72.15 ± 5.41 | 50.47 ± 4.38 | 57.27 ± 5.24 |
| (latency, s) | | | |

Memory Assay in T-maze

| | | | |
|---|---|---|---|
| Criteria achievement | 11/11 | 14/14 | 10/11 |
| (frequency/total n) | | | |
| Errors (frequency/total n) | 4/11 | 3/14 | 4/10 |

Finally in the Morris Water Maze test, during the cue learning task latency no meaningful differences were observed, probably due to the mice being young.

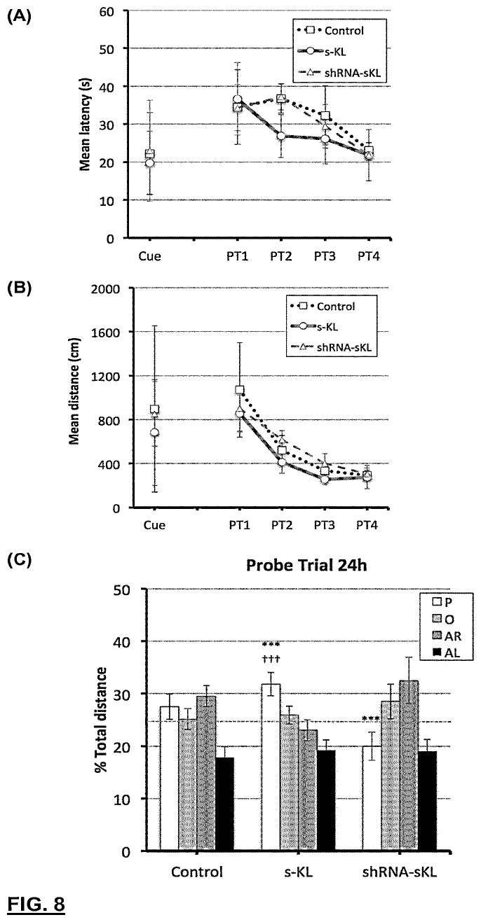

In the place task all groups showed a learning curve while training was performed, seen as a lower distance to get the platform. In relation to controls, m-KL and s-KL mice had a faster learning curve day by day. In the memory tests, 2 hours after the last cue assay, platform was removed and mice were evaluated to see if they could remember where the platform was. This was done determining the distance mice made in the square where the platform was (PTf), the distance in the opposite square (Opos PTf), in the right platform square (R PTf), and in the left platform square (L PTf). Data are not shown but all groups (null, and s-KL) were able to prioritize PTf square. On the other hand, if the test was performed 24 hours after the last cue test (long-term memory test), klotho treated groups were able to prioritize PTf square in a more efficient way than controls (p<0.001 for s-KL injected mice). Graphic data are depicted in FIG. 8, where percentage of total distance made in each of the squares is depicted for each mouse type.

Again, it could be concluded that as in older mice, klotho treatment improved memory skills (in this case long-term memory).

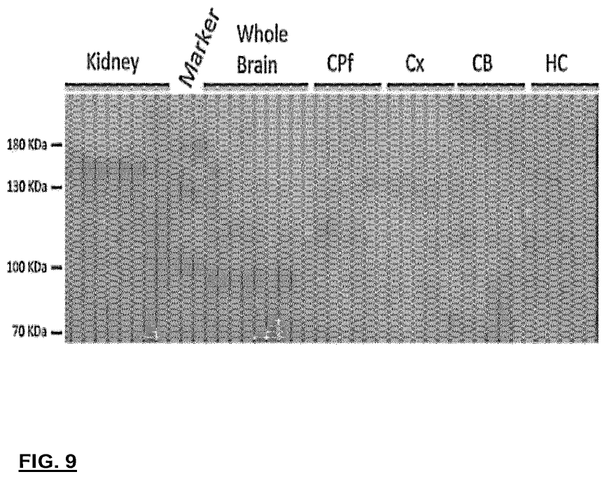

Visual perceptual learning and spatial reference learning and memory were assessed in the Morris Water Maze, following the same protocol as used previously. In the Place learning Task (PT) all groups showed the capacity to learn the task, reflected by the gradual reduction in latency to reach the platform (FIG. 8A) and in distance travelled to it (FIG. 8B). Finally, a memory probe test performed 24 hours after last training session allowed us to assess the effects of s-KL in long-term memory in 12 month old mice. FIG. 9C shows that mice overexpressing s-KL have a greater preference for the training quadrant (p=0.0003 versus Control and p<0.0001 versus shRNA-SKL). As before, shRNA-sKL treated animals showed the lowest preference for the training quadrant and random preference for the other quadrants (p<0.0001 versus control animals). We therefore conclude that, as in aged animals, elevation of s-KL levels enhances the ability to discriminate the quadrants, especially at long-term. Furthermore, silencing s-KL worsens the animals' performance, confirming it has a role in cognitive functions.

### Example 4. Detection of s-KL Protein Isoform With Specific Antibody

Protein extracts (15-25 g per sample) from tissue samples were run in denaturing acrylamide gels, and then electrotransferred to PDVF membranes (GE Healthcare). Membranes were blocked with TBS-T (20 mM Tris-HCl Ph 7.5, 150 mM NaCl, 0.2% Tween-20) containing 5% skimmed milk, and incubated with the primary K113 antibody. Detection was performed with an appropriate horseradish peroxidase-conjugated secondary antibody (EZBiolab, IN, USA) and enhanced chemiluminiscence reagent (GE Healthcare). The K113 antibody was used at 1/5,000 dilution; KM2076 antibody was used at 1/1000; polyclonal rabbit anti-actin antibody (Sigma A2066, USA) at 1/1,000; and secondary HRP-anti-lg antibody (Dako-Cytomation, P0399, Denmark) at 1/10,000.

As indicated above, the present invention results from inventors' determination in mouse wild-type brain tissue of the real expression at protein level (not only as mRNA) of a Klotho isoform, probably the splicing variant of mammal klotho protein.

This hypothesis results from an analysis of the klotho proteins detectable in wild-type mice tissues. Using the commercial antibody (KM2076, Cosmobio Japan) it could be determined in a Western Blot assay (FIG. 9) that in kidney the membrane isoform of 130 kDa was present, meanwhile in brain it only could be detected an isoform with a molecular weight similar to that of 70 KDa (the approximate weight of the KL1 domain of klotho or of the s-KL variant). The molecular weight of the brain detectable klotho isoform had a molecular weight between 70 KDa and 100 KDa, probably due to post-transcriptional modification in the amino acid sequence. FIG. 9 shows detection in whole brain, but also in prefrontal cortex (CPf), in cortex (Cx), cerebellum (CB), and hippocampus (HC).

Besides, in a parallel assay inventors determined with a self-made polyclonal antibody specific for mouse s-KL, if this isoform was in different brain sections. This specific polyclonal antibody (herewith named Ab K113) against s-KL was generated in rabbit by EZBiolab company (Carmel, USA) using the designed immunogenic peptide of SEQ ID NO: 11 (SPLTKPSVGLLLPH) as antigen. This sequence is not present either in m-KL o any p-KL, and it is for this reason that the antibody was specific for s-KL isoform.

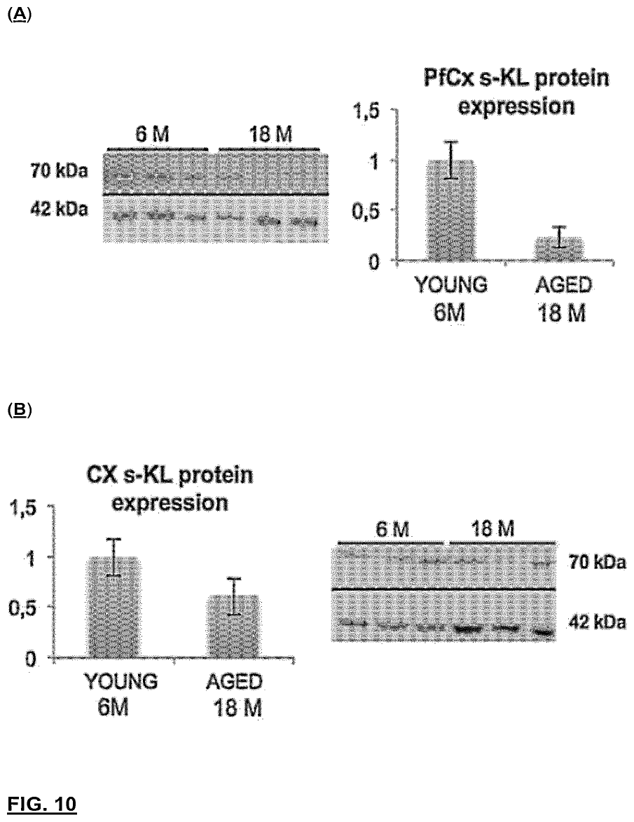

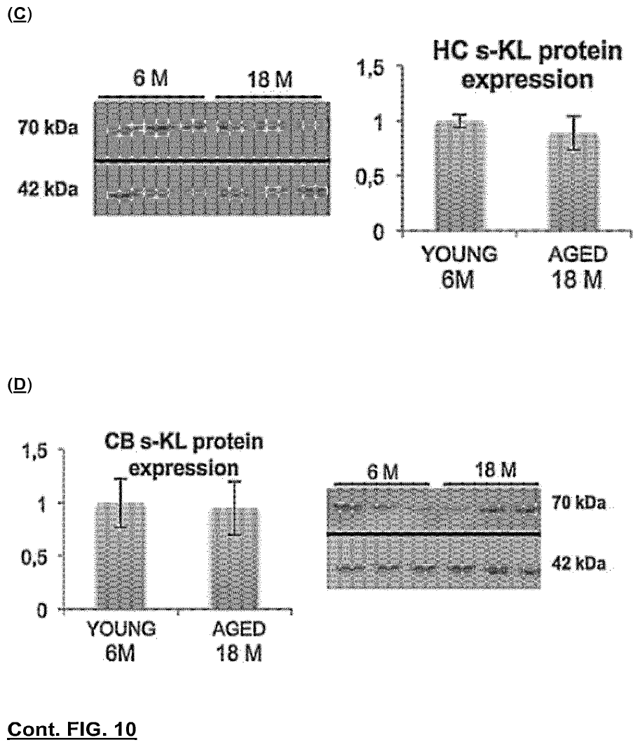

The levels of the s-KL protein were particularly analyzed in prefrontal cortex (PfCX s-KL), cortex (CX s-KL), hippocampus (HC s-KL) and cerebellum (CB s-KL) of 6 months old and 18 months-old mice using that K113 antibody. Data are depicted in FIG. 10, wherein Western-blot analysis is shown for each region together with densitometry assays (using Image J software). FIG. 10(A) depicts data from PfCX, FIG. 10(B) data from CX, FIG. 10(C) depicts data from HC and FIG. 10(D) data from CB.

All these data allow to conclude that the isoform of between 70-100 KDa detected by the commercial antibody in brain was likely s-KL, as the one specifically detected by K113 antibody of the inventors in the parallel assay of FIG. 10.

Therefore, if the variant of klotho protein that is endogenously expressed in brain is mainly s-KL, using it in the prevention and/or treatment of cognitive and/or behaviour impairment associated with aging, and/or with neurodegenerative and/or neuropathological diseases, supposes a real advantage. First, because from the data of Examples 2 and 3 of this invention it is derived that s-KL treatment supposes amelioration skills (memory and behaviour) in relation with non-treated animals.

Second, it is known from Kurosu, et al., “Regulation of fibroblast growth factor-23 signalling by klotho”, J Biol Chem-2006, vol 281 (10), pp.: 6120-3, that m-KL acts as a co-receptor of fibroblast growth factor receptor of FGF23 (FGF23R). This receptor is involved in calcium homeostasis. Therefore, by using s-KL, with similar effects than m-KL, this will avoid any interference with calcium homeostasis, since s-KL is not a co-receptor of FGF23R.

According to inventor's knowledge, this is the first time s-KL has been administered (in this case by gene therapy) to wild-type animals (mice). It has moreover been made plausible that this protein is therapeutically effective in terms of preserving and/or ameliorating cognitive and behaviour impairments associated with aging (in particular senile dementia) and with some neurodegenerative diseases, such as Alzheimer's disease (AD). In particular, therapeutically effective in preserving impairments in memory skills, such as memory losses and anxiety, all of them usually common in old people and in AD people.

In the case of neurodegenerative diseases and neuropathological diseases, it is highly recommendable proposing s-KL as accompanying treatment, if any.

### Example 5. Administration of s-KL and shRNA-sKL Expressing Vectors in Hippocampus Specifically Modifies Expression Levels of s-KL, but no m-KL

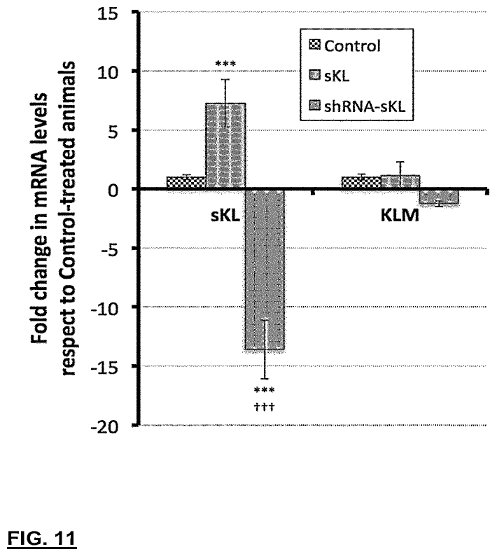

s-KL levels were quantified in the hippocampus of treated animals to determine whether the effects observed in cognition were induced by s-KL overexpression and/or s-KL inhibition. Hence, mRNA levels of s-KL in the AAV/s-KL injected group were increased 7.25±2.0 times with respect to animals injected with AAV-Control (p=0.035), while in AAV/shRNA-sKL animals, s-KL expression levels were reduced by 13.6±2.5 times (p=0.007) (FIG. 11). These changes were specific to the secreted Klotho isoform since expression of the m-KL transmembrane isoform was not affected either by s-KL overexpression (1.15+1.1 times vs control), nor by specific s-KL inhibition (−1.24±0.27 times vs control).

### STATISTICAL ANALYSIS

Values are presented as mean values+SEM. Statistical analyses and calculations were performed using the G-Stat version 2.0 and Prism 5.04 programs. Statistical analysis between individual groups was performed by two-tailed unpaired Student's t-test or one-way of variance ANOVA followed by Tukey post-hoc test. In all cases differences in means were considered statistically significant if p<0.05.

### REFERENCES CITED IN THE APPLICATION

Deary et al., “Klotho genotype and cognitive ability in childhood and old age in the same individuals”, Neurosci Lett-2005, vol. 378 (1), pp. 22-27.

Kuro-o, et al., Mutation of the mouse klotho gene leads to a syndrome resembling ageing. Nature-1997, vol. no. 390, pp.: 45-51.

Wang, Y. et al, “Current understanding of klotho”, Ageing Res Rev-2009, vol. no. 8, pp.: 43-511.

Matsumura et al., “Identification of the human klotho gene and its two transcripts encoding membrane and secreted Klotho protein”, Biochem Biophys Res Commun-1998, vol. No. 242, pp.: 626-630.

Dubai et al., “Life Extension Klotho Enhances Cognition”, Cell Reports-2014, vol. 7, pp.: 1065-1076.

Dubai et al., Life Extension factor Klotho Prevents Mortality and Enhances Cognition in hAPP Transgenic Mice”, The Journal of Neuroscience-2015, vol. 35/6, pp.: 2358-2371.

Kuang et al., “Klotho upregulation contributes to the neuroprotection of ligustilide in an Alzheimer's disease mouse model”, Neurobiology of Aging-2013, pp. 1-10.

Forster et al. “Vitamin D Receptor Controls Expression of the Anti-aging Klotho Gene in Mouse and Human Renal Cells” Biochem Biophys Res Commun. 2011 October 28; 414 (3): 557-562.

Shiraki-lida et al., “Structure of the mouse klotho gene and its two transcripts encoding membrane and secreted protein”, FEBS Letters—1998, vol 424, pp.: 6-10.

Imura et al., “Secreted Klotho protein in sera and CSF: implication for post-translational cleavage in release of Klotho protein from cell membrane”, FEBS Letters-2001, vol 565, pp.: 143-147.

Altschul, S. F., et al. “Gapped BLAST and PSI-BLAST: a new generation of proteina database search programms”, Nucleic Acids Research-1997, Vol. No. 25, pp.: 3389-3402.

http://www.ncbi.nlm.nih.gov/BLAST

Piedra et al., “Development of a rapid, robust, and universal picogreen-based method to titer adeno-associated vectors”, Hum Gene Ther Methods—2015, vol 26 (1), pp: 35-42; or doi: 10.1089/hgtb.2014.120 PMID: 25640021.—Hirt B et al., “Selective extraction of polyoma DNA from infected mouse cell cultures”. J Mol Biol-1967, vol 26, pp: 365-369.

Kurosu, et al., “Regulation of fibroblast growth factor-23 signaling by klotho”, J Biol Chem—-2006, vol 281 (10), pp.: 6120-3.

What is claimed is:

1. Secreted splicing variant of mammal klotho protein or nucleic acid sequence coding therefor, for use in the prevention and/or treatment of cognitive and/or behaviour impairment, and/or with neurodegenerative and/or neuropathological diseases in a mammal.

2.-16. (canceled)


SEQUENCE LISTING (19 sequences)

SEQ ID NO: 1 - protein, 549 aa, Homo sapiens
MPASAPPRRPRPPPPSLSLLLVLLGLGGRRLRAEPGDGAQTWARVSRPPAPEAAGLFQGTFPDGFLWAVGSAAYQTEGGWQQHGKGASIWDTFTHHPLAPPGDSRNASLPLGAPSPLQPATGDVASDSYNNVFRDTEALRELGVTHYRFSISWARVLPNGSAGVPNREGLRYYRRLLERLRELGVQPVVTLYHWDLPQRLQDAYGGWANRALADHFRDYAELCFRHFGGQVKYWITIDNPYVVAWHGYATGRLAPGIRGSPRLGYLVAHNLLLAHAKVWHLYNTSFRPTQGGQVSIALSSHWINPRRMTDHSIKECQKSLDFVLGWFAKPVFIDGDYPESMKNNLSSILPDFTESEKKFIKGTADFFALCFGPTLSFQLLDPHMKFRQLESPNLRQLLSWIDLEFNHPQIFIVENGWFVSGTTKRDDAKYMYYLKKFIMETLKAIKLDGVDVIGYTAWSLMDGFEWHRGYSIRRGLFYVDFLSQDKMLLPKSSALFYQKLIEKNGFPPLPENQPLEGTFPCDFAWGVVDNYIQVSQLTKPISSLTKPYH

SEQ ID NO: 2 - protein, 550 aa, Mus musculus
MLARAPPRRPPRLVLLRLLLLHLLLLALRARCLSAEPGQGAQTWARFARAPAPEAAGLLHDTFPDGFLWAVGSAAYQTEGGWRQHGKGASIWDTFTHHSGAAPSDSPIVVAPSGAPSPPLSSTGDVASDSYNNVYRDTEGLRELGVTHYRFSISWARVLPNGTAGTPNREGLRYYRRLLERLRELGVQPVVTLYHWDLPQRLQDTYGGWANRALADHFRDYAELCFRHFGGQVKYWITIDNPYVVAWHGYATGRLAPGVRGSSRLGYLVAHNLLLAHAKVWHLYNTSFRPTQGGRVSIALSSHWINPRRMTDYNIRECQKSLDFVLGWFAKPIFIDGDYPESMKNNLSSLLPDFTESEKRLIRGTADFFALSFGPTLSFQLLDPNMKFRQLESPNLRQLLSWIDLEYNHPPIFIVENGWFVSGTTKRDDAKYMYYLKKFIMETLKAIRLDGVDVIGYTAWSLMDGFEWHRGYSIRRGLFYVDFLSQDKELLPKSSALFYQKLIEDNGFPPLPENQPLEGTFPCDFAWGVVDNYVQVSPLTKPSVGLLLPH

SEQ ID NO: 3 - DNA, 3024 bp, synthetic construct
  misc_feature 1..3024: note = Gene construct comprising nucleotide sequence coding for Mus musculus klotho secreted splicing variant
  source 1..3024: mol_type = other DNA
tctagacatggctcgacagatctcaatattggccattagccatattattcattggttatatagcataaatcaatattggctattggccattgcatacgttgtatctatatcataatatgtacatttatattggctcatgtccaatatgaccgccatgttggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaacaactgcgatcgcccgccccgttgacgcaaatgggcggtaggcgtgtacggtgggaggtctatataagcagagctcgtttagtgaaccgtcagatcactagaagctttattgcggtagtttatcacagttaaattgctaacgcagtcagtgcttctgacacaacagtctcgaacttaagctgcagtgactctcttaaggtagccttgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccactcccagttcaattacagctcttaaggctagagtacttaatacgactcactataggctagcaatgctcgcccgcgcccctcctcgccgcccgccgcggctggtgctgctccgtttgctgttgctgcatctgctgctgctcgccctgcgcgcccgctgcctgagcgctgagccgggtcagggcgcgcagacctgggctcgcttcgcgcgcgctcctgccccagaggccgctggcctcctccacgacaccttccccgacggtttcctctgggcggtaggcagcgccgcctatcagaccgagggcggctggcgacagcacggcaaaggcgcgtccatctgggacactttcacccatcactctggggcggccccgtccgactccccgatcgtcgtggcgccgtcgggtgccccgtcgcctcccctgtcctccactggagatgtggccagcgatagttacaacaacgtctaccgcgacacagaggggctgcgcgaactgggggtcacccactaccgcttctccatatcgtgggcgcgggtgctccccaatggcaccgcgggcactcccaaccgcgaggggctgcgctactaccggcggctgctggagcggctgcgggagctgggcgtgcagccggtggttaccctgtaccattgggacctgccacagcgcctgcaggacacctatggcggatgggccaatcgcgccctggccgaccatttcagggattatgccgagctctgcttccgccacttcggtggtcaggtcaagtactggatcaccattgacaacccctacgtggtggcctggcacgggtatgccaccgggcgcctggccccgggcgtgaggggcagctccaggctcgggtacctggttgcccacaacctacttttggctcatgccaaagtctggcatctctacaacacctctttccgccccacacagggaggccgggtgtctatcgccttaagctcccattggatcaatcctcgaagaatgactgactataatatcagagaatgccagaagtctcttgactttgtgctaggctggtttgccaaacccatatttattgatggcgactacccagagagtatgaagaacaacctctcgtctcttctgcctgattttactgaatctgagaagaggctcatcagaggaactgctgacttttttgctctctccttcggaccaaccttgagctttcagctattggaccctaacatgaagttccgccaattggagtctcccaacctgaggcagcttctgtcttggatagatctggaatataaccaccctccaatatttattgtggaaaatggctggtttgtctcgggaaccaccaaaagggatgatgccaaatatatgtattatctcaagaagttcataatggaaaccttaaaagcaatcagactggatggggtcgacgtcattgggtacaccgcgtggtcgctcatggacggtttcgagtggcataggggctacagcatccggcgaggactcttctacgttgactttctgagtcaggacaaggagctgttgccaaagtcttcggccttgttctaccaaaagctgatagaggacaatggctttcctcctttacctgaaaaccagccccttgaagggacatttccctgtgactttgcttggggagttgttgacaactacgttcagctgagtcctttgacaaaacccagtgtcggcctcttgcttcctcactaagggccgcttccctttagtgagggttaatgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggta

SEQ ID NO: 4 - DNA, 3018 bp, synthetic construct
  misc_feature 1..3018: note = Gene construct comprising nucleotide sequence coding for Homo sapiens klotho secreted splicing variant
  source 1..3018: mol_type = other DNA
tctagacatggctcgacagatctcaatattggccattagccatattattcattggttatatagcataaatcaatattggctattggccattgcatacgttgtatctatatcataatatgtacatttatattggctcatgtccaatatgaccgccatgttggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaacaactgcgatcgcccgccccgttgacgcaaatgggcggtaggcgtgtacggtgggaggtctatataagcagagctcgtttagtgaaccgtcagatcactagaagctttattgcggtagtttatcacagttaaattgctaacgcagtcagtgcttctgacacaacagtctcgaacttaagctgcagtgactctcttaaggtagccttgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccactcccagttcaattacagctcttaaggctagagtacttaatacgactcactataggctagcaatgcccgccagcgccccgccgcgccgcccgcggccgccgccgccgtcgctgtcgctgctgctggtgctgctgggcctgggcggccgccgcctgcgtgcggagccgggcgacggcgcgcagacctgggcccgtgtctcgcggcctcctgcccccgaggccgcgggcctcttccagggcaccttccccgacggcttcctctgggccgtgggcagcgccgcctaccagaccgagggcggctggcagcagcacggcaagggtgcgtccatctgggacacgttcacccaccaccccctggcacccccgggagactcccggaacgccagtctgccgttgggcgccccgtcgccgctgcagcccgccaccggggacgtagccagcgacagctacaacaacgtcttccgcgacacggaggcgctgcgcgagctcggggtcactcactaccgcttctccatctcgtgggcgcgagtgctccccaatggcagcgcgggcgtccccaaccgcgaggggctgcgctactaccggcgcctgctggagcggctgcgggagctgggcgtgcagcccgtggtcaccctgtaccactgggacctgccccagcgcctgcaggacgcctacggcggctgggccaaccgcgccctggccgaccacttcagggattacgcggagctctgcttccgccacttcggcggtcaggtcaagtactggatcaccatcgacaacccctacgtggtggcctggcacggctacgccaccgggcgcctggcccccggcatccggggcagcccgcggctcgggtacctggtggcgcacaacctcctcctggctcatgccaaagtctggcatctctacaatacttctttccgtcccactcagggaggtcaggtgtccattgccctaagctctcactggatcaatcctcgaagaatgaccgaccacagcatcaaagaatgtcaaaaatctctggactttgtactaggttggtttgccaaacccgtatttattgatggtgactatcccgagagcatgaagaataacctttcatctattctgcctgattttactgaatctgagaaaaagttcatcaaaggaactgctgacttttttgctctttgctttggacccaccttgagttttcaacttttggaccctcacatgaagttccgccaattggaatctcccaacctgaggcaactgctttcctggattgaccttgaatttaaccatcctcaaatatttattgtggaaaatggctggtttgtctcagggaccaccaagagagatgatgccaaatatatgtattacctcaaaaagttcatcatggaaaccttaaaagccatcaagctggatggggtggatgtcatcgggtataccgcatggtccctcatggatggtttcgagtggcacagaggttacagcatcaggcgtggactcttctatgttgactttctaagccaggacaagatgttgttgccaaagtcttcagccttgttctaccaaaagctgatagagaaaaatggcttccctcctttacctgaaaatcagcccctagaagggacatttccctgtgactttgcttggggagttgttgacaactacattcagctgagtcctttgacaaaacccagtgtcggcctcttgcttcctcactaagggccgcttccctttagtgagggttaatgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggta

SEQ ID NO: 5 - DNA, 1650 bp, Homo sapiens
  source 1..1650: mol_type = other DNA
atgcccgccagcgccccgccgcgccgcccgcggccgccgccgccgtcgctgtcgctgctgctggtgctgctgggcctgggcggccgccgcctgcgtgcggagccgggcgacggcgcgcagacctgggcccgtgtctcgcggcctcctgcccccgaggccgcgggcctcttccagggcaccttccccgacggcttcctctgggccgtgggcagcgccgcctaccagaccgagggcggctggcagcagcacggcaagggtgcgtccatctgggacacgttcacccaccaccccctggcacccccgggagactcccggaacgccagtctgccgttgggcgccccgtcgccgctgcagcccgccaccggggacgtagccagcgacagctacaacaacgtcttccgcgacacggaggcgctgcgcgagctcggggtcactcactaccgcttctccatctcgtgggcgcgagtgctccccaatggcagcgcgggcgtccccaaccgcgaggggctgcgctactaccggcgcctgctggagcggctgcgggagctgggcgtgcagcccgtggtcaccctgtaccactgggacctgccccagcgcctgcaggacgcctacggcggctgggccaaccgcgccctggccgaccacttcagggattacgcggagctctgcttccgccacttcggcggtcaggtcaagtactggatcaccatcgacaacccctacgtggtggcctggcacggctacgccaccgggcgcctggcccccggcatccggggcagcccgcggctcgggtacctggtggcgcacaacctcctcctggctcatgccaaagtctggcatctctacaatacttctttccgtcccactcagggaggtcaggtgtccattgccctaagctctcactggatcaatcctcgaagaatgaccgaccacagcatcaaagaatgtcaaaaatctctggactttgtactaggttggtttgccaaacccgtatttattgatggtgactatcccgagagcatgaagaataacctttcatctattctgcctgattttactgaatctgagaaaaagttcatcaaaggaactgctgacttttttgctctttgctttggacccaccttgagttttcaacttttggaccctcacatgaagttccgccaattggaatctcccaacctgaggcaactgctttcctggattgaccttgaatttaaccatcctcaaatatttattgtggaaaatggctggtttgtctcagggaccaccaagagagatgatgccaaatatatgtattacctcaaaaagttcatcatggaaaccttaaaagccatcaagctggatggggtggatgtcatcgggtataccgcatggtccctcatggatggtttcgagtggcacagaggttacagcatcaggcgtggactcttctatgttgactttctaagccaggacaagatgttgttgccaaagtcttcagccttgttctaccaaaagctgatagagaaaaatggcttccctcctttacctgaaaatcagcccctagaagggacatttccctgtgactttgcttggggagttgttgacaactacattcaagtaagtcagctgacaaaaccaatcagcagtctcaccaagccctatcactag

SEQ ID NO: 6 - DNA, 1653 bp, Mus musculus
  source 1..1653: mol_type = other DNA
atgctagcccgcgcccctcctcgccgcccgccgcggctggtgctgctccgtttgctgttgctgcatctgctgctgctcgccctgcgcgcccgctgcctgagcgctgagccgggtcagggcgcgcagacctgggctcgcttcgcgcgcgctcctgccccagaggccgctggcctcctccacgacaccttccccgacggtttcctctgggcggtaggcagcgccgcctatcagaccgagggcggctggcgacagcacggcaaaggcgcgtccatctgggacactttcacccatcactctggggcggccccgtccgactccccgatcgtcgtggcgccgtcgggtgccccgtcgcctcccctgtcctccactggagatgtggccagcgatagttacaacaacgtctaccgcgacacagaggggctgcgcgaactgggggtcacccactaccgcttctccatatcgtgggcgcgggtgctccccaatggcaccgcgggcactcccaaccgcgaggggctgcgctactaccggcggctgctggagcggctgcgggagctgggcgtgcagccggtggttaccctgtaccattgggacctgccacagcgcctgcaggacacctatggcggatgggccaatcgcgccctggccgaccatttcagggattatgccgagctctgcttccgccacttcggtggtcaggtcaagtactggatcaccattgacaacccctacgtggtggcctggcacgggtatgccaccgggcgcctggccccgggcgtgaggggcagctccaggctcgggtacctggttgcccacaacctacttttggctcatgccaaagtctggcatctctacaacacctctttccgccccacacagggaggccgggtgtctatcgccttaagctcccattggatcaatcctcgaagaatgactgactataatatcagagaatgccagaagtctcttgactttgtgctaggctggtttgccaaacccatatttattgatggcgactacccagagagtatgaagaacaacctctcgtctcttctgcctgattttactgaatctgagaagaggctcatcagaggaactgctgacttttttgctctctccttcggaccaaccttgagctttcagctattggaccctaacatgaagttccgccaattggagtctcccaacctgaggcagcttctgtcttggatagatctggaatataaccaccctccaatatttattgtggaaaatggctggtttgtctcgggaaccaccaaaagggatgatgccaaatatatgtattatctcaagaagttcataatggaaaccttaaaagcaatcagactggatggggtcgacgtcattgggtacaccgcgtggtcgctcatggacggtttcgagtggcataggggctacagcatccggcgaggactcttctacgttgactttctgagtcaggacaaggagctgttgccaaagtcttcggccttgttctaccaaaagctgatagaggacaatggctttcctcctttacctgaaaaccagccccttgaagggacatttccctgtgactttgcttggggagttgttgacaactacgttcaagtaagtcctttgacaaaacccagtgtcggcctcttgcttcctcactaa

SEQ ID NO: 7 - DNA, 20 bp, synthetic construct
  misc_feature 1..20: note = forward primer
  source 1..20: mol_type = other DNA
ttcaaacccggaagtctttg

SEQ ID NO: 8 - DNA, 20 bp, synthetic construct
  misc_feature 1..20: note = Reverse primer
  source 1..20: mol_type = other DNA
ccaggcagacgttcacatta

SEQ ID NO: 9 - DNA, 20 bp, synthetic construct
  misc_feature 1..20: note = Forward primer
  source 1..20: mol_type = other DNA
tggctttcctcctttacctg

SEQ ID NO: 10 - DNA, 18 bp, synthetic construct
  misc_feature 1..18: note = Reverse primer
  source 1..18: mol_type = other DNA
gccgacactgggttttgt

SEQ ID NO: 11 - protein, 14 aa, Mus musculus
SPLTKPSVGLLLPH

SEQ ID NO: 12 - DNA, 5421 bp, synthetic construct
  misc_feature 1..5421: note = Plasmid pGG2
  regulatory 206..955: note = promoter - CMV IE enhancer/promoter; regulatory_class = promoter
  misc_feature 1095..1227: note = Intron - Intron from pCl-neo
  misc_feature 1227..1334: note = Multiple Cloning Site (MCS)
  regulatory 1369..1590: note = polyA_signal - SV40 late poly A signal; regulatory_class = polyA_signal_sequence
  source 1..5421: mol_type = other DNA
cagcagctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctagacatggctcgacagatctcaatattggccattagccatattattcattggttatatagcataaatcaatattggctattggccattgcatacgttgtatctatatcataatatgtacatttatattggctcatgtccaatatgaccgccatgttggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaacaactgcgatcgcccgccccgttgacgcaaatgggcggtaggcgtgtacggtgggaggtctatataagcagagctcgtttagtgaaccgtcagatcactagaagctttattgcggtagtttatcacagttaaattgctaacgcagtcagtgcttctgacacaacagtctcgaacttaagctgcagtgactctcttaaggtagccttgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccactcccagttcaattacagctcttaaggctagagtacttaatacgactcactataggctagcctcgacctcgagacgcgtgatatcggatcccggccggcggccgcttccctttagtgagggttaatgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatccgataagggactagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgccagctggcgtaatagcgaagaggcccgcaccgatcgcccttcccaacagttgcgcagcctgaatggcgaatggaattccagacgattgagcgtcaaaatgtaggtatttccatgagcgtttttccgttgcaatggctggcggtaatattgttctggatattaccagcaaggccgatagtttgagttcttctactcaggcaagtgatgttattactaatcaaagaagtattgcgacaacggttaatttgcgtgatggacagactcttttactcggtggcctcactgattataaaaacacttctcaggattctggcgtaccgttcctgtctaaaatccctttaatcggcctcctgtttagctcccgctctgattctaacgaggaaagcacgttatacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtctacaatttaaatatttgcttatacaatcttcctgtttttggggcttttctgattatcaaccggggtacatatgattgacatgctagttttacgattaccgttcatcgattctcttgtttgctccagactctcaggcaatgacctgatagcctttgtagagacctctcaaaaatagctaccctctccggcatgaatttatcagctagaacggttgaatatcatattgatggtgatttgactgtctccggcctttctcacccgtttgaatctttacctacacattactcaggcattgcatttaaaatatatgagggttctaaaaatttttatccttgcgttgaaataaaggcttctcccgcaaaagtattacagggtcataatgtttttggtacaaccgatttagctttatgctctgaggctttattgcttaattttgctaattctttgccttgcctgtatgatttattggatgttggaatcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatg

SEQ ID NO: 13 - DNA, 8335 bp, synthetic construct
  misc_feature 1..8335: note = pGG2 plasmid comprising sequences of CMV IE promoter and the sequence coding for mouse m-KL (pGG2-m-KL)
  source 1..8335: mol_type = other DNA
cagcagctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctagacatggctcgacagatctcaatattggccattagccatattattcattggttatatagcataaatcaatattggctattggccattgcatacgttgtatctatatcataatatgtacatttatattggctcatgtccaatatgaccgccatgttggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaacaactgcgatcgcccgccccgttgacgcaaatgggcggtaggcgtgtacggtgggaggtctatataagcagagctcgtttagtgaaccgtcagatcactagaagcttgatatcgaattcgcccttgataatcattgctcgtggggcggcgggagcgggggtgggcaccgcgtagggagggcggcggggcgcgggcatataggggcgcggcgcggtgccctcccggctcccgcagcatgctagcccgcgcccctcctcgccgcccgccgcggctggtgctgctccgtttgctgttgctgcatctgctgctgctcgccctgcgcgcccgctgcctgagcgctgagccgggtcagggcgcgcagacctgggctcgcttcgcgcgcgctcctgccccagaggccgctggcctcctccacgacaccttccccgacggtttcctctgggcggtaggcagcgccgcctatcagaccgagggcggctggcgacagcacggcaaaggcgcgtccatctgggacactttcacccatcactctggggcggccccgtccgactccccgatcgtcgtggcgccgtcgggtgccccgtcgcctcccctgtcctccactggagatgtggccagcgatagttacaacaacgtctaccgcgacacagaggggctgcgcgaactgggggtcacccactaccgcttctccatatcgtgggcgcgggtgctccccaatggcaccgcgggcactcccaaccgcgaggggctgcgctactaccggcggctgctggagcggctgcgggagctgggcgtgcagccggtggttaccctgtaccattgggacctgccacagcgcctgcaggacacctatggcggatgggccaatcgcgccctggccgaccatttcagggattatgccgagctctgcttccgccacttcggtggtcaggtcaagtactggatcaccattgacaacccctacgtggtggcctggcacgggtatgccaccgggcgcctggccccgggcgtgaggggcagctccaggctcgggtacctggttgcccacaacctacttttggctcatgccaaagtctggcatctctacaacacctctttccgccccacacagggaggccgggtgtctatcgccttaagctcccattggatcaatcctcgaagaatgactgactataatatcagagaatgccagaagtctcttgactttgtgctaggctggtttgccaaacccatatttattgatggcgactacccagagagtatgaagaacaacctctcgtctcttctgcctgattttactgaatctgagaagaggctcatcagaggaactgctgacttttttgctctctccttcggaccaaccttgagctttcagctattggaccctaacatgaagttccgccaattggagtctcccaacctgaggcagcttctgtcttggatagatctggaatataaccaccctccaatatttattgtggaaaatggctggtttgtctcgggaaccaccaaaagggatgatgccaaatatatgtattatctcaagaagttcataatggaaaccttaaaagcaatcagactggatggggtcgacgtcattgggtacaccgcgtggtcgctcatggacggtttcgagtggcataggggctacagcatccggcgaggactcttctacgttgactttctgagtcaggacaaggagctgttgccaaagtcttcggccttgttctaccaaaagctgatagaggacaatggctttcctcctttacctgaaaaccagccccttgaagggacatttccctgtgactttgcttggggagttgttgacaactacgttcaagtggacactactctctctcagtttactgacccgaatgtctatctgtgggatgtgcatcacagtaagaggcttattaaagtagacggggttgtagccaagaagagaaaaccttactgtgttgatttctctgccatccggcctcagataaccttacttcgagaaatgcgggtcacccactttcgcttctccctggactgggccctgatcttgcctctgggtaaccagacccaagtgaaccacacggttctgcacttctaccgctgcatgatcagcgagctggtgcacgccaacatcactccagtggtggccctgtggcagccagcagccccgcaccaaggcctgccacatgcccttgcaaaacatggggcctgggagaacccgcacactgctctggcgtttgcagactacgcaaacctgtgttttaaagagttgggtcactgggtcaatctctggatcaccatgaacgagccaaacacacggaacatgacctatcgtgccgggcaccacctcctgagagcccatgccttggcttggcatctgtacgatgacaagtttagggcggctcagaaaggcaaaatatccatcgccttgcaggctgactggatagaaccggcctgccctttctctcaaaatgacaaagaagtggccgagagagttttggaatttgatataggctggctggcagagcctatttttggttccggagattatccacgtgtgatgagggactggctgaaccaaaaaaacaattttcttttgccctatttcaccgaagatgaaaaaaagctagtccggggttcctttgacttcctggcggtgagtcattacaccaccattctggtagactgggaaaaggaggatccgatgaaatacaacgattacttggaggtacaggagatgactgacatcacatggctcaactctcccagtcaggtggcagtggtgccttgggggctgcgcaaagtgctcaactggctaaggttcaagtacggagacctcccgatgtatgtgacagccaatggaatcgatgatgacccccacgccgagcaagactcactgaggatctattatattaagaattatgtgaatgaggctctgaaagcctacgtgttggacgacatcaacctttgtggctactttgcgtattcacttagtgatcgctcagctcccaagtctggcttttatcgatatgctgcgaatcagtttgagcccaaaccatctatgaaacattacaggaaaattattgacagcaatggcttcctgggttctggaacactgggaaggttttgtccagaagaatacactgtgtgcaccgaatgtggattttttcaaacccggaagtctttgctggtcttcatctcgtttcttgtttttacttttattatttctcttgctctcatttttcactactccaagaaaggccagagaagttataagtaatgtgaacgtctgcctggccattcgctttgggatcaagatgtacacgccgtcagccgtttgcacctctctgtgttgtgagccgcattccacacatttcgattctagagcggccgcttccctttagtgagggttaatgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatccgataagggactagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgccagctggcgtaatagcgaagaggcccgcaccgatcgcccttcccaacagttgcgcagcctgaatggcgaatggaattccagacgattgagcgtcaaaatgtaggtatttccatgagcgtttttccgttgcaatggctggcggtaatattgttctggatattaccagcaaggccgatagtttgagttcttctactcaggcaagtgatgttattactaatcaaagaagtattgcgacaacggttaatttgcgtgatggacagactcttttactcggtggcctcactgattataaaaacacttctcaggattctggcgtaccgttcctgtctaaaatccctttaatcggcctcctgtttagctcccgctctgattctaacgaggaaagcacgttatacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtctacaatttaaatatttgcttatacaatcttcctgtttttggggcttttctgattatcaaccggggtacatatgattgacatgctagttttacgattaccgttcatcgattctcttgtttgctccagactctcaggcaatgacctgatagcctttgtagagacctctcaaaaatagctaccctctccggcatgaatttatcagctagaacggttgaatatcatattgatggtgatttgactgtctccggcctttctcacccgtttgaatctttacctacacattactcaggcattgcatttaaaatatatgagggttctaaaaatttttatccttgcgttgaaataaaggcttctcccgcaaaagtattacagggtcataatgtttttggtacaaccgatttagctttatgctctgaggctttattgcttaattttgctaattctttgccttgcctgtatgatttattggatgttggaatcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatg

SEQ ID NO: 14 - DNA, 5640 bp, synthetic construct
  misc_feature 1..5640: note = synthetic plasmid pUC57-KL (GenScript, USA)
  source 1..5640: mol_type = other DNA
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcttctagaatgctcgcccgcgcccctcctcgccgcccgccgcggctggtgctgctccgtttgctgttgctgcatctgctgctgctcgccctgcgcgcccgctgcctgagcgctgagccgggtcagggcgcgcagacctgggctcgcttcgcgcgcgctcctgccccagaggccgctggcctcctccacgacaccttccccgacggtttcctctgggcggtaggcagcgccgcctatcagaccgagggcggctggcgacagcacggcaaaggcgcgtccatctgggacactttcacccatcactctggggcggccccgtccgactccccgatcgtcgtggcgccgtcgggtgccccgtcgcctcccctgtcctccactggagatgtggccagcgatagttacaacaacgtctaccgcgacacagaggggctgcgcgaactgggggtcacccactaccgcttctccatatcgtgggcgcgggtgctccccaatggcaccgcgggcactcccaaccgcgaggggctgcgctactaccggcggctgctggagcggctgcgggagctgggcgtgcagccggtggttaccctgtaccattgggacctgccacagcgcctgcaggacacctatggcggatgggccaatcgcgccctggccgaccatttcagggattatgccgagctctgcttccgccacttcggtggtcaggtcaagtactggatcaccattgacaacccctacgtggtggcctggcacgggtatgccaccgggcgcctggccccgggcgtgaggggcagctccaggctcgggtacctggttgcccacaacctacttttggctcatgccaaagtctggcatctctacaacacctctttccgccccacacagggaggccgggtgtctatcgccttaagctcccattggatcaatcctcgaagaatgactgactataatatcagagaatgccagaagtctcttgactttgtgctaggctggtttgccaaacccatatttattgatggcgactacccagagagtatgaagaacaacctctcgtctcttctgcctgattttactgaatctgagaagaggctcatcagaggaactgctgacttttttgctctctccttcggaccaaccttgagctttcagctattggaccctaacatgaagttccgccaattggagtctcccaacctgaggcagcttctgtcttggatagatctggaatataaccaccctccaatatttattgtggaaaatggctggtttgtctcgggaaccaccaaaagggatgatgccaaatatatgtattatctcaagaagttcataatggaaaccttaaaagcaatcagactggatggggtcgacgtcattgggtacaccgcgtggtcgctcatggacggtttcgagtggcataggggctacagcatccggcgaggactcttctacgttgactttctgagtcaggacaaggagctgttgccaaagtcttcggccttgttctaccaaaagctgatagaggacaatggctttcctcctttacctgaaaaccagccccttgaagggacatttccctgtgactttgcttggggagttgttgacaactacgttcagctgagtcctttgacaaaacccagtgtcggcctcttgcttcctcactaagggcccagagactgaagctttacgtagacactactctctctcagtttactgacccgaatgtctatctgtgggatgtgcatcacagtaagaggcttattaaagtagacggggttgtagccaaggctagcaaaccttactgtgttgatttctctgccatccggcctcagataaccttacttcgagaaatgcgggtcacccactttcgcttctccctggactgggcgctgatcttgcctctgggtaaccagacccaagtgaaccacacggttctgcacttctaccgctgcatgatcagcgagctggtgcacgccaacatcactccagtggtggccctgtggcagccagcagccccgcaccaaggcctgccacatgcccttgcaaaacatggggcctgggagaacccgcacactgctctggcgtttgcagactacgcaaacctgtgttttaaagagttgggtcactgggtcaatctctggatcaccatgaacgagccaaacacacggaacatgacctatcgtgccgggcaccacctcctgagagcccatgccttggcttggcatctgtacgatgacaagtttagggcggctcagaaaggcaaaatatccatcgccttgcaggctgactggatagaaccggcctgccctttctctcaaaatgacaaagaagtggccgagagagttttggaatttgatataggctggctggcagagcctatttttggttccggagattatccacgtgtgatgagggactggctgaaccaaaaaaacaattttcttttgccctatttcaccgaagatgaaaaaaagctagtccggggttcctttgacttcctggcggtgagtcattacaccaccattctggtagactgggaaaaggaggatccgatgaaatacaacgattacttggaggtacaggagatgactgacatcacatggctcaactctcccagtcaggtggcagtggtgccttgggggctgcgcaaagtgctcaactggctaaggttcaagtacggagacctcccgatgtatgtgacagccaatggaatcgatgatgacccccacgccgagcaagactcactgaggatctattatattaagaattatgtgaatgaggctctgaaagcctacgtgttggacgacatcaacctttgtggctactttgcgtattcacttagtgatcgctcagctcccaagtctggcttttatcgatatgctgcgaatcagtttgagcccaaaccatctatgaaacattacaggaaaattattgacagcaatggcttcctgggttctggaacactgggaaggttttgtccagaagaataagcggccgcgcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 15 - DNA, 5931 bp, synthetic construct
  misc_feature 1..5931: note = pGG2 plasmid comprising sequences of CMV IE promoter and the sequence coding for mouse s-KL (pGG2-s-KL)
  source 1..5931: mol_type = other DNA
cagcagctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctagaatgctcgcccgcgcccctcctcgccgcccgccgcggctggtgctgctccgtttgctgttgctgcatctgctgctgctcgccctgcgcgcccgctgcctgagcgctgagccgggtcagggcgcgcagacctgggctcgcttcgcgcgcgctcctgccccagaggccgctggcctcctccacgacaccttccccgacggtttcctctgggcggtaggcagcgccgcctatcagaccgagggcggctggcgacagcacggcaaaggcgcgtccatctgggacactttcacccatcactctggggcggccccgtccgactccccgatcgtcgtggcgccgtcgggtgccccgtcgcctcccctgtcctccactggagatgtggccagcgatagttacaacaacgtctaccgcgacacagaggggctgcgcgaactgggggtcacccactaccgcttctccatatcgtgggcgcgggtgctccccaatggcaccgcgggcactcccaaccgcgaggggctgcgctactaccggcggctgctggagcggctgcgggagctgggcgtgcagccggtggttaccctgtaccattgggacctgccacagcgcctgcaggacacctatggcggatgggccaatcgcgccctggccgaccatttcagggattatgccgagctctgcttccgccacttcggtggtcaggtcaagtactggatcaccattgacaacccctacgtggtggcctggcacgggtatgccaccgggcgcctggccccgggcgtgaggggcagctccaggctcgggtacctggttgcccacaacctacttttggctcatgccaaagtctggcatctctacaacacctctttccgccccacacagggaggccgggtgtctatcgccttaagctcccattggatcaatcctcgaagaatgactgactataatatcagagaatgccagaagtctcttgactttgtgctaggctggtttgccaaacccatatttattgatggcgactacccagagagtatgaagaacaacctctcgtctcttctgcctgattttactgaatctgagaagaggctcatcagaggaactgctgacttttttgctctctccttcggaccaaccttgagctttcagctattggaccctaacatgaagttccgccaattggagtctcccaacctgaggcagcttctgtcttggatagatctggaatataaccaccctccaatatttattgtggaaaatggctggtttgtctcgggaaccaccaaaagggatgatgccaaatatatgtattatctcaagaagttcataatggaaaccttaaaagcaatcagactggatggggtcgacgtcattgggtacaccgcgtggtcgctcatggacggtttcgagtggcataggggctacagcatccggcgaggactcttctacgttgactttctgagtcaggacaaggagctgttgccaaagtcttcggccttgttctaccaaaagctgatagaggacaatggctttcctcctttacctgaaaaccagccccttgaagggacatttccctgtgactttgcttggggagttgttgacaactacgttcagctgagtcctttgacaaaacccagtgtcggcctcttgcttcctcactaagggccgcttccctttagtgagggttaatgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatccgataagggactagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgccagctggcgtaatagcgaagaggcccgcaccgatcgcccttcccaacagttgcgcagcctgaatggcgaatggaattccagacgattgagcgtcaaaatgtaggtatttccatgagcgtttttccgttgcaatggctggcggtaatattgttctggatattaccagcaaggccgatagtttgagttcttctactcaggcaagtgatgttattactaatcaaagaagtattgcgacaacggttaatttgcgtgatggacagactcttttactcggtggcctcactgattataaaaacacttctcaggattctggcgtaccgttcctgtctaaaatccctttaatcggcctcctgtttagctcccgctctgattctaacgaggaaagcacgttatacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtctacaatttaaatatttgcttatacaatcttcctgtttttggggcttttctgattatcaaccggggtacatatgattgacatgctagttttacgattaccgttcatcgattctcttgtttgctccagactctcaggcaatgacctgatagcctttgtagagacctctcaaaaatagctaccctctccggcatgaatttatcagctagaacggttgaatatcatattgatggtgatttgactgtctccggcctttctcacccgtttgaatctttacctacacattactcaggcattgcatttaaaatatatgagggttctaaaaatttttatccttgcgttgaaataaaggcttctcccgcaaaagtattacagggtcataatgtttttggtacaaccgatttagctttatgctctgaggctttattgcttaattttgctaattctttgccttgcctgtatgatttattggatgttggaatcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatg

SEQ ID NO: 16 - DNA, 18689 bp, synthetic construct
  misc_feature 1..18689: note = Plasmid pXX6
  source 1..18689: mol_type = other DNA
ttattttggattgaagccaatatgataatgagggggtggagtttgtgacgtggcgcggggcgtgggaacggggcgggtgacgtagtagtgtggcggaagtgtgatgttgcaagtgtggcggaacacatgtaagcgacggatgtggcaaaagtgacgtttttggtgtgcgcggatccacaggacgggtgtggtcgccatgatcgcgtagtcgatagtggctccaagtagcgaagcgagcaggactgggcggcggccaaagcggtcggacagtgctccgagaacgggtgcgcatagaaattgcatcaacgcatatagcgctagcagcacgccatagtgactggcgatgctgtcggaatggacgatatcccgcaagaggcccggcagtaccggcataaccaagcctatgcctacagcatccagggtgacggtgccgaggatgacgatgagcgcattgttagatttcatacacggtgcctgactgcgttagcaatttaactgtgataaactaccgcattaaagcttatcgataagcttgatatcgaattcctgcagcccgggggatccactagttctagagcggccgccaccgcggtggagctccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctgggtaccgggccccccctcgaggtcgacagaagcaccatgtccttgggtccggcctgctgaatgcgcaggcggtcggccatgccccaggcttcgttttgacatcggcgcaggtctttgtagtagtcttgcatgagcctttctaccggcacttcttcttctccttcctcttgtcctgcatctcttgcatctatcgctgcggcggcggcggagtttggccgtaggtggcgccctcttcctcccatgcgtgtgaccccgaagcccctcatcggctgaagcagggctaggtcggcgacaacgcgctcggctaatatggcctgctgcacctgcgtgagggtagactggaagtcatccatgtccacaaagcggtggtatgcgcccgtgttgatggtgtaagtgcagttggccataacggaccagttaacggtctggtgacccggctgcgagagctcggtgtacctgagacgcgagtaagccctcgagtcaaatacgtagtcgttgcaagtccgcaccaggtactggtatcccaccaaaaagtgcggcggcggctggcggtagaggggccagcgtagggtggccggggctccgggggcgagatcttccaacataaggcgatgatatccgtagatgtacctggacatccaggtgatgccggcggcggtggtggaggcgcgcggaaagtcgcggacgcggttccagatgttgcgcagcggcaaaaagtgctccatggtcgggacgctctggccggtcaggcgcgcgcaatcgttgacgctctagcgtgcaaaaggagagcctgtaagcgggcactcttccgtggtctggtggataaattcgcaagggtatcatggcggacgaccggggttcgagccccgtatccggccgtccgccgtgatccatgcggttaccgcccgcgtgtcgaacccaggtgtgcgacgtcagacaacgggggagtgctccttttggcttccttccaggcgcggcggctgctgcgctagcttttttggccactggccgcgcgcagcgtaagcggttaggctggaaagcgaaagcattaagtggctcgctccctgtagccggagggttattttccaagggttgagtcgcgggacccccggttcgagtctcggaccggccggactgcggcgaacgggggtttgcctccccgtcatgcaagaccccgcttgcaaattcctccggaaacagggacgagccccttttttgcttttcccagatgcatccggtgctgcggcagatgcgcccccctcctcagcagcggcaagagcaagagcagcggcagacatgcagggcaccctcccctcctcctaccgcgtcaggaggggcgacatccgcggttgacgcggcagcagatggtgattacgaacccccgcggcgccgggcccggcactacctggacttggaggagggcgagggcctggcgcggctaggagcgccctctcctgagcggtacccaagggtgcagctgaagcgtgatacgcgtgaggcgtacgtgccgcggcagaacctgtttcgcgaccgcgagggagaggagcccgaggagatgcgggatcgaaagttccacgcagggcgcgagctgcggcatggcctgaatcgcgagcggttgctgcgcgaggaggactttgagcccgacgcgcgaaccgggattagtcccgcgcgcgcacacgtggcggccgccgacctggtaaccgcatacgagcagacggtgaaccaggagattaactttcaaaaaagctttaacaaccacgtgcgtacgcttgtggcgcgcgaggaggtggctataggactgatgcatctgtgggactttgtaagcgcgctggagcaaaacccaaatagcaagccgctcatggcgcagctgttccttatagtgcagcacagcagggacaacgaggcattcagggatgcgctgctaaacatagtagagcccgagggccgctggctgctcgatttgataaacatcctgcagagcatagtggtgcaggagcgcagcttgagcctggctgacaaggtggccgccatcaactattccatgcttagcctgggcaagttttacgcccgcaagatataccataccccttacgttcccatagacaaggaggtaaagatcgaggggttctacatgcgcatggcgctgaaggtgcttaccttgagcgacgacctgggcgtttatcgcaacgagcgcatccacaaggccgtgagcgtgagccggcggcgcgagctcagcgaccgcgagctgatgcacagcctgcaaagggccctggctggcacgggcagcggcgatagagaggccgagtcctactttgacgcgggcgctgacctgcgctgggccccaagccgacgcgccctggaggcagctggggccggacctgggctggcggtggcacccgcgcgcgctggcaacgtcggcggcgtggaggaatatgacgaggacgatgagtacgagccagaggacggcgagtactaagcggtgatgtttctgatcagatgatgcaagacgcaacggacccggcggtgcgggcggcgctgcagagccagccgtccggccttaactccacggacgactggcgccaggtcatggaccgcatcatgtcgctgactgcgcgcaatcctgacgcgttccggcagcagccgcaggccaaccggctctccgcaattctggaagcggtggtcccggcgcgcgcaaaccccacgcacgagaaggtgctggcgatcgtaaacgcgctggccgaaaacagggccatccggcccgacgaggccggcctggtctacgacgcgctgcttcagcgcgtggctcgttacaacagcggcaacgtgcagaccaacctggaccggctggtgggggatgtgcgcgaggccgtggcgcagcgtgagcgcgcgcagcagcagggcaacctgggctccatggttgcactaaacgccttcctgagtacacagcccgccaacgtgccgcggggacaggaggactacaccaactttgtgagcgcactgcggctaatggtgactgagacaccgcaaagtgaggtgtaccagtctgggccagactattttttccagaccagtagacaaggcctgcagaccgtaaacctgagccaggctttcaaaaacttgcaggggctgtggggggtgcgggctcccacaggcgaccgcgcgaccgtgtctagcttgctgacgcccaactcgcgcctgttgctgctgctaatagcgcccttcacggacagtggcagcgtgtcccgggacacatacctaggtcacttgctgacactgtaccgcgaggccataggtcaggcgcatgtggacgagcatactttccaggagattacaagtgtcagccgcgcgctggggcaggaggacacgggcagcctggaggcaaccctaaactacctgctgaccaaccggcggcagaagatcccctcgttgcacagtttcgcaccctttggcgcatcccattctccagtaactttatgtccatgggcgcactcacagacctgggccaaaaccttctctacgccaactccgcccacgcgctagacatgacttttgaggtggatcccatggacgagcccacccttctttatgttttgtttgaagtctttgacgtggtccgtgtgcaccggccgcaccgcggcgtcatcgaaaccgtgtacctgcgcacgcccttctcggccggcaacgccacaacataaagaagcaagcaacatcaacaacagctgccgccatgggctccagtgagcaggaactgaaagccattgtcaaagatcttggttgtgggccatattttttgggcacctatgacaagcgctttccaggctttgtttctccacacaagctcgcctgcgccatagtcaatacggccggtcgcgagactgggggcgtacactggatggcctttgcctggaacccgcactcaaaaacatgctacctctttgagccctttggcttttctgaccagcgactcaagcaggtttaccagtttgagtacgagtcactcctgcgccgtagcgccattgcttcttcccccgaccgctgtataacgctggaaaagtccacccaaagcgtacaggggcccaactcggccgcctgtggactattctgctgcatgtttctccacgcctttgccaactggccccaaactcccatggatcacaaccccaccatgaaccttattaccggggtacccaactccatgctcaacagtccccaggtacagcccaccctgcgtcgcaaccaggaacagctctacagcttcctggagcgccactcgccctacttccgcagccacagtgcgcagattaggagcgccacttctttttgtcacttgaaaaacatgtaaaaataatgtactagagacactttcaataaaggcaaatgcttttatttgtacactctcgggtgattatttacccccacccttgccgtctgcgccgtttaaaaatcaaaggggttctgccgcgcatcgctatgcgccactggcagggacacgttgcgatactggtgtttagtgctccacttaaactcaggcacaaccatccgcggcagctcggtgaagttttcactccacaggctgcgcaccatcaccaacgcgtttagcaggtcgggcgccgatatcttgaagtcgcagttggggcctccgccctgcgcgcgcgagttgcgatacacagggttgcagcactggaacactatcagcgccgggtggtgcacgctggccagcacgctcttgtcggagatcagatccgcgtccaggtcctccgcgttgctcagggcgaacggagtcaactttggtagctgccttcccaaaaagggcgcgtgcccaggctttgagttgcactcgcaccgtagtggcatcaaaaggtgaccgtgcccggtctgggcgttaggatacagcgcctgcataaaagccttgatctgcttaaaagccacctgagcctttgcgccttcagagaagaacatgccgcaagacttgccggaaaactgattggccggacaggccgcgtcgtgcacgcagcaccttgcgtcggtgttggagatctgcaccacatttcggccccaccggttcttcacgatcttggccttgctagactgctccttcagcgcgcgctgcccgttttcgctcgtcacatccatttcaatcacgtgctccttatttatcataatgcttccgtgtagacacttaagctcgccttcgatctcagcgcagcggtgcagccacaacgcgcagcccgtgggctcgtgatgcttgtaggtcacctctgcaaacgactgcaggtacgcctgcaggaatcgccccatcatcgtcacaaaggtcttgttgctggtgaaggtcagctgcaacccgcggtgctcctcgttcagccaggtcttgcatacggccgccagagcttccacttggtcaggcagtagtttgaagttcgcctttagatcgttatccacgtggtacttgtccatcagcgcgcgcgcagcctccatgcccttctcccacgcagacacgatcggcacactcagcgggttcatcaccgtaatttcactttccgcttcgctgggctcttcctcttcctcttgcgtccgcataccacgcgccactgggtcgtcttcattcagccgccgcactgtgcgcttacctcctttgccatgcttgattagcaccggtgggttgctgaaacccaccatttgtagcgccacatcttctctttcttcctcgctgtccacgattacctctggtgatggcgggcgctcgggcttgggagaagggcgcttctttttcttcttgggcgcaatggccaaatccgccgccgaggtcgatggccgcgggctgggtgtgcgcggcaccagcgcgtcttgtgatgagtcttcctcgtcctcggactcgatacgccgcctcatccgcttttttgggggcgcccggggaggcggcggcgacggggacggggacgacacgtcctccatggttgggggacgtcgcgccgcaccgcgtccgcgctcgggggtggtttcgcgctgctcctcttcccgactggccatttccttctcctataggcagaaaaagatcatggagtcagtcgagaagaaggacagcctaaccgccccctctgagttcgccaccaccgcctccaccgatgccgccaacgcgcctaccaccttccccgtcgaggcacccccgcttgaggaggaggaagtgattatcgagcaggacccaggttttgtaagcgaagacgacgaggaccgctcagtaccaacagaggataaaaagcaagaccaggacaacgcagaggcaaacgaggaacaagtcgggcggggggacgaaaggcatggcgactacctagatgtgggagacgacgtgctgttgaagcatctgcagcgccagtgcgccattatctgcgacgcgttgcaagagcgcagcgatgtgcccctcgccatagcggatgtcagccttgcctacgaacgccacctattctcaccgcgcgtaccccccaaacgccaagaaaacggcacatgcgagcccaacccgcgcctcaacttctaccccgtatttgccgtgccagaggtgcttgccacctatcacatctttttccaaaactgcaagatacccctatcctgccgtgccaaccgcagccgagcggacaagcagctggccttgcggcagggcgctgtcatacctgatatcgcctcgctcaacgaagtgccaaaaatctttgagggtcttggacgcgacgagaagcgcgcggcaaacgctctgcaacaggaaaacagcgaaaatgaaagtcactctggagtgttggtggaactcgagggtgacaacgcgcgcctagccgtactaaaacgcagcatcgaggtcacccactttgcctacccggcacttaacctaccccccaaggtcatgagcacagtcatgagtgagctgatcgtgcgccgtgcgcagcccctggagagggatgcaaatttgcaagaacaaacagaggagggcctacccgcagttggcgacgagcagctagcgcgctggcttcaaacgcgcgagcctgccgacttggaggagcgacgcaaactaatgatggccgcagtgctcgttaccgtggagcttgagtgcatgcagcggttctttgctgacccggagatgcagcgcaagctagaggaaacattgcactacacctttcgacagggctacgtacgccaggcctgcaagatctccaacgtggagctctgcaacctggtctcctaccttggaattttgcacgaaaaccgccttgggcaaaacgtgcttcattccacgctcaagggcgaggcgcgccgcgactacgtccgcgactgcgtttacttatttctatgctacacctggcagacggccatgggcgtttggcagcagtgcttggaggagtgcaacctcaaggagctgcagaaactgctaaagcaaaacttgaaggacctatggacggccttcaacgagcgctccgtggccgcgcacctggcggacatcattttccccgaacgcctgcttaaaaccctgcaacagggtctgccagacttcaccagtcaaagcatgttgcagaactttaggaactttatcctagagcgctcaggaatcttgcccgccacctgctgtgcacttcctagcgactttgtgcccattaagtaccgcgaatgccctccgccgctttggggccactgctaccttctgcagctagccaactaccttgcctaccactctgacataatggaagacgtgagcggtgacggtctactggagtgtcactgtcgctgcaacctatgcaccccgcaccgctccctggtttgcaattcgcagctgcttaacgaaagtcaaattatcggtacctttgagctgcagggtccctcgcctgacgaaaagtccgcggctccggggttgaaactcactccggggctgtggacgtcggcttaccttcgcaaatttgtacctgaggactaccacgcccacgagattaggttctacgaagaccaatcccgcccgccaaatgcggagcttaccgcctgcgtcattacccagggccacattcttggccaattgcaagccatcaacaaagcccgccaagagtttctgctacgaaagggacggggggtttacttggacccccagtccggcgaggagctcaacccaatccccccgccgccgcagccctatcagcagcagccgcgggcccttgcttcccaggatggcacccaaaaagaagctgcagctgccgccgccacccacggacgaggaggaatactgggacagtcaggcagaggaggttttggacgaggaggaggaggacatgatggaagactgggagagcctagacgaggaagcttccgaggtcgaagaggtgtcagacgaaacaccgtcaccctcggtcgcattcccctcgccggcgccccagaaatcggcaaccggttccagcatggctacaacctccgctcctcaggcgccgccggcactgcccgttcgccgacccaaccgtagatgggacaccactggaaccagggccggtaagtccaagcagccgccgccgttagcccaagagcaacaacagcgccaaggctaccgctcatggcgcgggcacaagaacgccatagttgcttgcttgcaagactgtgggggcaacatctccttcgcccgccgctttcttctctaccatcacggcgtggccttcccccgtaacatcctgcattactaccgtcatctctacagcccatactgcaccggcggcagcggcagcggcagcaacagcagcggccacacagaagcaaaggcgaccggatagcaagactctgacaaagcccaagaaatccacagcggcggcagcagcaggaggaggagcgctgcgtctggcgcccaacgaacccgtatcgacccgcgagcttagaaacaggatttttcccactctgtatgctatatttcaacagagcaggggccaagaacaagagctgaaaataaaaaacaggtctctgcgatccctcacccgcagctgcctgtatcacaaaagcgaagatcagcttcggcgcacgctggaagacgcggaggctctcttcagtaaatactgcgcgctgactcttaaggactagtttcgcgccctttctcaaatttaagcgcgaaaactacgtcatctccagcggccacacccggcgccagcacctgtcgtcagcgccattatgagcaaggaaattcccacgccctacatgtggagttaccagccacaaatgggacttgcggctggagctgcccaagactactcaacccgaataaactacatgagcgcgggaccccacatgatatcccgggtcaacggaatccgcgcccaccgaaaccgaattctcttggaacaggcggctattaccaccacacctcgtaataaccttaatccccgtagttggcccgctgccctggtgtaccaggaaagtcccgctcccaccactgtggtacttcccagagacgcccaggccgaagttcagatgactaactcaggggcgcagcttgcgggcggctttcgtcacagggtgcggtcgcccgggcagggtataactcacctgacaatcagagggcgaggtattcagctcaacgacgagtcggtgagctcctcgcttggtctccgtccggacgggacatttcagatcggcggcgccggccgtccttcattcacgcctcgtcaggcaatcctaactctgcagacctcgtcctctgagccgcgctctggaggcattggaactctgcaatttattgaggagtttgtgccatcggtctactttaaccccttctcgggacctcccggccactatccggatcaatttattcctaactttgacgcggtaaaggactcggcggacggctacgactgaatgttaagtggagaggcagagcaactgcgcctgaaacacctggtccactgtcgccgccacaagtgctttgcccgcgactccggtgagttttgctactttgaattgcccgaggatcatatcgagggcccggcgcacggcgtccggcttaccgcccagggagagcttgcccgtagcctgattcgggagtttacccagcgccccctgctagttgagcgggacaggggaccctgtgttctcactgtgatttgcaactgtcctaaccttggattacatcaaccctttaactaataaaaaaaaataataaagcatcacttacttaaaatcagttagcaaatttctgtccagtttattcagcagcacctccttgccctcctcccagctctggtattgcagcttcctcctggctgcaaactttctccacaatctaaatggaatgtcagtttcctcctgttcctgtccatccgcacccactatcttcatgttgttgcagatgaagcgcgcaagaccgtctgaagataccttcaaccccgtgtatccatatgacacggaaaccggtcctccaactgtgccttttcttactcctccctttgtatcccccaatgggtttcaagagagtccccctggggtactctctttgcgcctatccgaacctctagttacctccaatggcatgcttgcgctcaaaatgggcaacggcctctctctggacgaggccggcaaccttacctcccaaaatgtaaccactgtgagcccacctctcaaaaaaaccaagtcaaacataaacctggaaatatctgcacccctcacagttacctcagaagccctaactgtggctgccgccgcacctctaatggtcgcgggcaacacactcaccatgcaatcacaggccccgctaaccgtgcacgactccaaacttagcattgccacccaaggacccctcacagtgtcagaaggaaagctagccctgcaaacatcaggccccctcaccaccaccgatagcagtacccttactatcactgcctcaccccctctaactactgccactggtagcttgggcattgacttgaaagagcccatttatacacaaaatggaaaactaggactaaagtacggggctcctttgcatgtaacagacgacctaaacactttgaccgtagcaactggtccaggtgtgactattaataatacttccttgcaaactaaagttactggagccttgggttttgattcacaaggcaatatgcaacttaatgtagcaggaggactaaggattgattctcaaaacagacgccttatacttgatgttagttatccgtttgatgctcaaaaccaactaaatctaagactaggacagggccctctttttataaactcagcccacaacttggatattaactacaacaaaggcctttacttgtttacagcttcaaacaattccaaaaagcttgaggttaacctaagcactgccaaggggttgatgtttgacgctacagccatagccattaatgcaggagatgggcttgaatttggttcacctaatgcaccaaacacaaatcccctcaaaacaaaaattggccatggcctagaatttgattcaaacaaggctatggttcctaaactaggaactggccttagttttgacagcacaggtgccattacagtaggaaacaaaaataatgataagctaactttgtggaccacaccagctccatctcctaactgtagactaaatgcagagaaagatgctaaactcactttggtcttaacaaaatgtggcagtcaaatacttgctacagtttcagttttggctgttaaaggcagtttggctccaatatctggaacagttcaaagtgctcatcttattataagatttgacgaaaatggagtgctactaaacaattccttcctggacccagaatattggaactttagaaatggagatcttactgaaggcacagcctatacaaacgctgttggatttatgcctaacctatcagcttatccaaaatctcacggtaaaactgccaaaagtaacattgtcagtcaagtttacttaaacggagacaaaactaaacctgtaacactaaccattacactaaacggtacacaggaaacaggagacacaactccaagtgcatactctatgtcattttcatgggactggtctggccacaactacattaatgaaatatttgccacatcctcttacactttttcatacattgcccaagaataaagaatcgtttgtgttatgtttcaacgtgtttatttttcaattgcagaaaatttcaagtcatttttcattcagtagtatagccccaccaccacatagcttatacagatcaccgtaccttaatcaaactcacagaaccctagtattcaacctgccacctccctcccaacacacagagtacacagtcctttctccccggctggccttaaaaagcatcatatcatgggtaacagacatattcttaggtgttatattccacacggtttcctgtcgagccaaacgctcatcagtgatattaataaactccccgggcagctcacttaagttcatgtcgctgtccagctgctgagccacaggctgctgtccaacttgcggttgcttaacgggcggcgaaggagaagtccacgcctacatgggggtagagtcataatcgtgcatcaggatagggcggtggtgctgcagcagcgcgcgaataaactgctgccgccgccgctccgtcctgcaggaatacaacatggcagtggtctcctcagcgatgattcgcaccgcccgcagcataaggcgccttgtcctccgggcacagcagcgcaccctgatctcacttaaatcagcacagtaactgcagcacagcaccacaatattgttcaaaatcccacagtgcaaggcgctgtatccaaagctcatggcggggaccacagaacccacgtggccatcataccacaagcgcaggtagattaagtggcgacccctcataaacacgctggacataaacattacctcttttggcatgttgtaattcaccacctcccggtaccatataaacctctgattaaacatggcgccatccaccaccatcctaaaccagctggccaaaacctgcccgccggctatacactgcagggaaccgggactggaacaatgacagtggagagcccaggactcgtaaccatggatcatcatgctcgtcatgatatcaatgttggcacaacacaggcacacgtgcatacacttcctcaggattacaagctcctcccgcgttagaaccatatcccagggaacaacccattcctgaatcagcgtaaatcccacactgcagggaagacctcgcacgtaactcacgttgtgcattgtcaaagtgttacattcgggcagcagcggatgatcctccagtatggtagcgcgggtttctgtctcaaaaggaggtagacgatccctactgtacggagtgcgccgagacaaccgagatcgtgttggtcgtagtgtcatgccaaatggaacgccggacgtagtcatatttcctgaagcaaaaccaggtgcgggcgtgacaaacagatctgcgtctccggtctcgccgcttagatcgctctgtgtagtagttgtagtatatccactctctcaaagcatccaggcgccccctggcttcgggttctatgtaaactccttcatgcgccgctgccctgataacatccaccaccgcagaataagccacacccagccaacctacacattcgttctgcgagtcacacacgggaggagcgggaagagctggaagaaccatgtttttttttttattccaaaagattatccaaaacctcaaaatgaagatctattaagtgaacgcgctcccctccggtggcgtggtcaaactctacagccaaagaacagataatggcatttgtaagatgttgcacaatggcttccaaaaggcaaacggccctcacgtccaagtggacgtaaaggctaaacccttcagggtgaatctcctctataaacattccagcaccttcaaccatgcccaaataattctcatctcgccaccttctcaatatatctctaagcaaatcccgaatattaagtccggccattgtaaaaatctgctccagagcgccctccaccttcagcctcaagcagcgaatcatgattgcaaaaattcaggttcctcacagacctgtataagattcaaaagcggaacattaacaaaaataccgcgatcccgtaggtcccttcgcagggccagctgaacataatcgtgcaggtctgcacggaccagcgcggccacttccccgccaggaaccttgacaaaagaacccacactgattatgacacgcatactcggagctatgctaaccagcgtagccccgatgtaagctttgttgcatgggcggcgatataaaatgcaaggtgctgctcaaaaaatcaggcaaagcctcgcgcaaaaaagaaagcacatcgtagtcatgctcatgcagataaaggcaggtaagctccggaaccaccacagaaaaagacaccatttttctctcaaacatgtctgcgggtttctgcataaacacaaaataaaataacaaaaaaacatttaaacattagaagcctgtcttacaacaggaaaaacaacccttataagcataagacggactacggccatgccggcgtgaccgtaaaaaaactggtcaccgtgattaaaaagcaccaccgacagctcctcggtcatgtccggagtcataatgtaagactcggtaaacacatcaggttgattcatcggtcagtgctaaaaagcgaccgaaatagcccgggggaatacatacccgcaggcgtagagacaacattacagcccccataggaggtataacaaaattaataggagagaaaaacacataaacacctgaaaaaccctcctgcctaggcaaaatagcaccctcccgctccagaacaacatacagcgcttcacagcggcagcctaacagtcagccttaccagtaaaaaagaaaacctattaaaaaaacaccactcgacacggcaccagctcaatcagtcacagtgtaaaaaagggccaagtgcagagcgagtatatataggactaaaaaatgacgtaacggttaaagtccacaaaaaacacccagaaaaccgcacgcgaacctacgcccagaaacgaaagccaaaaaacccacaacttcctcaaatcgtcacttccgttttcccacgttacgtaacttcccattttaagaaaactacaattcccaacacatacaagttactccgccctaaaacctacgtcacccgccccgttcccacgccccgcgccacgtcacaaactccaccccctcattatcatattggcttcaatccaaaataaggtatattattgatgatg

SEQ ID NO: 17 - DNA, 7335 bp, synthetic construct
  misc_feature 1..7335: note = Plasmid pREp2Cap10
  source 1..7335: mol_type = other DNA
gattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggctcttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgatttaaatcaggtatggctgccgatggttatcttccagattggctcgaggacaacctctctgagggcattcgcgagtggtgggacttgaaacctggagccccgaaacccaaagccaaccagcaaaagcaggacgacggccggggtctggtgcttcctggctacaagtacctcggacccttcaacggactcgacaagggggagcccgtcaacgcggcggacgcagcggccctcgagcacgacaaggcctacgaccagcagctcaaagcgggtgacaatccgtacctgcggtataaccacgccgacgccgagtttcaggagcgtctgcaagaagatacgtcttttgggggcaacctcgggcgagcagtcttccaggccaagaagcgggttctcgaacctctcggtctggttgaggaaggcgctaagacggctcctggaaagaagagaccggtagagccatcaccccagcgttctccagactcctctacgggcatcggcaagaaaggccagcagcccgcgaaaaagagactcaactttgggcagactggcgactcagagtcagtgcccgaccctcaaccaatcggagaaccccccgcaggcccctctggtctgggatctggtacaatggctgcaggcggtggcgctccaatggcagacaataacgaaggcgccgacggagtgggtagttcctcaggaaattggcattgcgattccacatggctgggcgacagagtcatcaccaccagcacccgaacctgggccctccccacctacaacaaccacctctacaagcaaatctccaacgggacttcgggaggaagcaccaacgacaacacctacttcggctacagcaccccctgggggtattttgactttaacagattccactgccacttctcaccacgtgactggcagcgactcatcaacaacaactggggattccggcccaagagactcaacttcaagctcttcaacatccaggtcaaggaggtcacgcagaatgaaggcaccaagaccatcgccaataaccttaccagcacgattcaggtctttacggactcggaataccagctcccgtacgtcctcggctctgcgcaccagggctgcctgcctccgttcccggcggacgtcttcatgattcctcagtacgggtacctgactctgaacaatggcagtcaggccgtgggccgttcctccttctactgcctggagtactttccttctcaaatgctgagaacgggcaacaactttgagttcagctaccagtttgaggacgtgccttttcacagcagctacgcgcacagccaaagcctggaccggctgatgaaccccctcatcgaccagtacctgtactacctgtctcggactcagtccacgggaggtaccgcaggaactcagcagttgctattttctcaggccgggcctaataacatgtcggctcaggccaaaaactggctacccgggccctgctaccggcagcaacgcgtctccacgacactgtcgcaaaataacaacagcaactttgcctggaccggtgccaccaagtatcatctgaatggcagagactctctggtaaatcccggtgtcgctatggcaacccacaaggacgacgaagagcgattttttccgtccagcggagtcttaatgtttgggaaacagggagctggaaaagacaacgtggactatagcagcgttatgctaaccagtgaggaagaaattaaaaccaccaacccagtggccacagaacagtacggcgtggtggccgataacctgcaacagcaaaacgccgctcctattgtaggggccgtcaacagtcaaggagccttacctggcatggtctggcagaaccgggacgtgtacctgcagggtcctatctgggccaagattcctcacacggacggaaactttcatccctcgccgctgatgggaggctttggactgaaacacccgcctcctcagatcctgattaagaatacacctgttcccgcggatcctccaactaccttcagtcaagctaagctggcgtcgttcatcacgcagtacagcaccggacaggtcagcgtggaaattgaatgggagctgcagaaagaaaacagcaaacgctggaacccagagattcaatacacttccaactactacaaatctacaaatgtggactttgctgttaacacagatggcacttattctgagcctcgccccatcggcacccgttacctcacccgtaatctgtaattgcttgttaatcaataaaccggttgattcgtttcagttgaactttggtctctgcatatggcgaattcgtttaaacctgcaggactagagtcctgtattagaggtcacgtgagtgttttgcgacattttgcgacaccatgtggtcacgctgggtatttaagcccgagtgagcacgcagggtctccattttgaagcgggaggtttgaacgcgcagccgccaagccgaattctgcagatatccatcacactggcggccgctcgactagagcggccgccaccgcggtggagctccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattgggtaccgggccccccctcgatcgaggtcgacggtatcgggggagctcgcagggtctccattttgaagcgggaggtttgaacgcgcagccgccatgccggggttttacgagattgt

SEQ ID NO: 18 - DNA, 22 bp, synthetic construct
  misc_feature 1..22: note = Forward primer
  source 1..22: mol_type = other DNA
tacataacttacggtaaatggc

SEQ ID NO: 19 - DNA, 21 bp, synthetic construct
  misc_feature 1..21: note = Reverse primer
  source 1..21: mol_type = other DNA
aaagtccctattggcgttact